(19) United States
(12) Patent Application Publication
(10) Pub. No.: US 2024/0225991 A1
(43) Pub. Date: Jul. 11, 2024

(54) COMPOSITION COMPRISING AT LEAST ONE SILICONE OF FORMULA (I), AT LEAST ONE SILICONE OF FORMULA (II) AND AT LEAST ONE COLORING AGENT

(71) Applicant: L'OREAL, Paris (FR)

(72) Inventors: Marine GERARDIN-PROTAT, SAINT-OUEN (FR); Alexis LIARD, SAINT-OUEN (FR); Arnaud BONNAMY, SAINT-OUEN (FR); Zhibo LIU, AULNAY-SOUS-BOIS (FR)

(21) Appl. No.: 18/287,145

(22) PCT Filed: Dec. 15, 2021
(86) PCT No.: PCT/EP2021/085866

§ 371 Date: Oct. 16, 2023

(30) Foreign Application Priority Data
Dec. 17, 2020 (FR) 2013551
Dec. 17, 2020 (FR) 2013554
Dec. 17, 2020 (FR) 2013555

(51) Int. Cl. A61K 8/892; A61K 8/19; A61K 8/29; A61K 8/58; A61K 8/893; A61K 8/898; A61Q 5/06

(52) U.S. Cl. CPC A61K 8/892; A61K 8/19; A61K 8/29; A61K 8/585; A61K 8/893; A61K 8/898; A61Q 5/065; A61K 2800/43; A61K 2800/432; A61K 2800/884

(57) ABSTRACT

The present invention relates to a composition for treating keratin fibers such as the hair, comprising at least one silicone of formula (I), at least one silicone of formula (II) and at least one coloring agent chosen from pigments, direct dyes and mixtures thereof.

### TECHNICAL FIELD OF THE INVENTION

The present invention relates to a composition for treating keratin fibers such as the hair, comprising at least one silicone of formula (I) as described below, at least one silicone of formula (II) as described below, at least one coloring agent chosen from pigments, direct dyes and mixtures thereof and optionally at least one metallic compound.

The present invention also relates to a method for treating keratin fibers such as the hair, comprising a step of applying to the keratin fibers a composition (A) comprising at least one metallic compound; and a step of applying to the keratin fibers a composition (B) comprising at least one silicone of formula (I), and at least one silicone of formula (II), the composition (A) and/or the composition (B) comprising at least one coloring agent chosen from pigments, direct dyes and mixtures thereof.

### CONTEXT OF THE INVENTION

In the field of dyeing of keratin fibers, in particular human keratin fibers, it is already known practice to dye keratin fibers via various techniques starting from direct dyes or pigments for nonpermanent dyeing or from dye precursors for permanent dyeing.

There are essentially three types of process for dyeing the hair:
a) “permanent” dyeing, the function of which is to afford a substantial modification to the natural color and which employs oxidation dyes which penetrate into the hair fiber and forms the dye via an oxidative condensation process;
b) nonpermanent, semipermanent or direct dyeing, which does not employ the oxidative condensation process and withstands four or five shampoo washes; it consists in dyeing keratin fibers with dye compositions containing direct dyes;
c) temporary dyeing, which gives rise to a modification to the natural color of the head of hair which remains from one shampooing operation to the next, and which serves to enhance or correct a shade which has already been obtained. It can also be likened to a “makeup” process.

For this last type of dyeing, it is known practice to use colored polymers formed by grafting one or more dyes of azo, triphenylmethane, azine, indoamine or anthraquinone nature to a polymer chain. These colored polymers are not entirely satisfactory, in particular as regards the homogeneity of the coloring obtained and its resistance, not to mention the problems associated with their manufacture and in particular with their reproducibility.

Another dyeing method consists in using pigments. Specifically, the use of pigment on the surface of keratin fibers generally makes it possible to obtain visible colorings on dark hair, since the surface pigment masks the natural color of the fiber. However, the colorings obtained via this dyeing method have the drawback of having poor resistance to shampoo washing and also to external agents such as sebum, perspiration, brushing and/or rubbing.

In addition, there are no effective makeup-removing compositions for removing this type of temporary dye composition when it is persistent with respect to shampoo washing.

Furthermore, such a pigment-based dye may require the application of high heat and/or the application of heat over a prolonged time to the keratin fibers in order to accelerate the formation of the colored coating on the keratin fibers and to improve the persistence of the colored coating with respect to shampoo washing and to external agents.

However, this requires the use of a particular heating tool which makes the application of the composition on the keratin fibers more complex.

The need thus remains to have available a composition for treating keratin fibers, in particular the hair, which exhibits the advantage of obtaining a homogeneous and smooth colored coating on the hairs, while forming a covering which is persistent toward shampooing operations and to the various attacking factors to which the hair may be subjected, such as brushing and/or rubbing actions, without degrading the hair and without applying excessive heat. There is also the need to be able to eliminate this colored coating when so desired.

The applicant has discovered, surprisingly, that all of these objectives can be achieved by means of the composition according to the present invention.

### SUMMARY OF THE INVENTION

The present invention relates to a composition for treating keratin fibers such as the hair, comprising:
a) at least one silicone of formula (I) as described below,
b) at least one silicone of formula (II) as described below, and
c) at least one coloring agent chosen from pigments, direct dyes and mixtures thereof.

The present invention also relates to a method for treating, in particular for dyeing, keratin fibers, such as the hair, wherein the composition according to the invention as described below is applied to said fibers.

The present invention also relates to the use of the composition as described below for treating keratin fibers such as the hair, in particular for dyeing keratin fibers such as the hair.

The present invention also relates to a method for treating keratin fibers such as the hair, comprising:
(i) a step of applying to the keratin fibers a composition (A) comprising at least one metallic compound; and
(ii) a step of applying to the keratin fibers a composition (B) comprising:
   at least one silicone of formula (I) as described below, and
   at least one silicone of formula (II) as described below,
the composition (A) and/or the composition (B) comprising at least one coloring agent chosen from pigments, direct dyes and mixtures thereof,
it being understood that steps i) and ii) are performed successively i), then ii) or else ii), then i).

Through the use of this composition, colored coatings are obtained on the hair that make it possible to obtain a coloring that is visible on all types of hair in a manner that is persistent with respect to shampoo washing, while at the same time preserving the physical qualities of the keratin fibers. Such a coating may be resistant to the external attacking factors to which the hair may be subjected, such as blow-drying and perspiration. It makes it possible in particular to obtain a smooth and uniform deposit on the keratin fibers.

Furthermore, such a composition does not require the application of excessive heat to the keratin fibers.

Advantageously, the colored coating thus obtained can be readily eliminated by means of a makeup-removing composition.

For the purposes of the present invention, the expression “coloring that is persistent with respect to shampoo washing” is understood to mean that the coloring obtained persists after one shampoo wash, preferably after three shampoo washes.

For the purposes of the present invention, the expression “application of excessive heat to the keratin fibers”, is understood to mean an application to the keratin fibers of heat strictly above 110° C., for example heat between 110° C. and 220° C. over a relatively short application time, for example of less than 10 minutes, or else the application to the keratin fibers of heat between 45° C. and 110° C. for a relatively long application time, for example greater than or equal to 30 minutes.

The expression “at least one” means one or more.

The expression “keratin fibers” is understood particularly to mean human keratin fibers, such as the hair, eyelashes, eyebrows and body hair, preferentially the hair, eyebrows and eyelashes, more preferentially still the hair.

For the purposes of the present invention, the expression “hair” means the hair of the head. It does not refer to eyelashes, eyebrows and body hair.

For the purposes of the present invention, the expression “successive steps” is understood to mean steps performed in the indicated order.

For the purposes of the present invention, the term “silicone” denotes any organosilicon polymer or oligomer of linear or cyclic, branched or crosslinked structure, of variable molecular weight, obtained by polymerization and/or by polycondensation of suitably functionalized silanes, and constituted essentially of a repetition of main units in which the silicon atoms are linked together via oxygen atoms (siloxane bond —Si—O—Si—), optionally substituted hydrocarbon-based radicals being directly bonded via a carbon atom to said silicon atoms.

For the purposes of the present invention and unless otherwise indicated,
an “alkyl” radical denotes a linear or branched, saturated radical containing, for example, from 1 to 30 carbon atoms;
an “aminoalkyl” radical denotes an alkyl radical as defined previously, said alkyl radical comprising an NH₂ group;
a “dialkylamino” radical denotes an amino radical substituted with two alkyl groups as defined previously, the two alkyl groups possibly being identical or different, the two alkyl groups possibly further being able to form, with the nitrogen atom that bears them, a 5- or 8-membered heterocycle;
a “(di)alkylamino” radical denotes an aminoalkyl radical or a dialkylamino radical as defined previously;
a “cycloalkyl” radical denotes a saturated cyclic hydrocarbon-based group comprising from 1 to 3 rings, preferably 2 rings, and comprising from 3 to 20 carbon atoms, preferably between 5 and 10 carbon atoms, such as cyclopentyl, cyclohexyl, cycloheptyl, norbornyl, or isobornyl, the cycloalkyl radical possibly being substituted with one or more (C1-C4)alkyl groups such as methyl;
an “aryl” radical is a monocyclic or bicyclic, fused or non-fused, unsaturated cyclic aromatic radical comprising from 6 to 12 carbon atoms; preferably, the aryl group comprises 1 ring containing 6 carbon atoms, such as phenyl;
an “alkoxy” radical denotes an “alkyl-oxy” radical with “alkyl” as defined previously;
an “aralkyl” radical denotes an “alkyl” radical as defined previously substituted with an “aryl” radical as defined previously;
an “acyl” radical denotes a radical R-CO with R representing the alkyl radical as defined previously.

Unless otherwise indicated, when compounds are mentioned in the present application, this also includes the optical isomers thereof, the geometrical isomers thereof, the tautomers thereof, or the salts thereof, alone or as a mixture.

The invention is not limited to the examples illustrated. The features of the various examples may in particular be combined within variants which are not illustrated.

The composition for treating keratin fibers according to the invention is preferably a composition for dyeing keratin fibers such as the hair.

The treatment method according to the invention is a method for dyeing keratin fibers such as the hair.

Preferably, the composition (A) and/or the composition (B) are compositions for treating keratin fibers such as the hair and more specifically compositions for dyeing keratin fibers such as the hair.

### DETAILED DESCRIPTION OF THE INVENTION

Composition according to the invention Silicone of formula (I):

The composition according to the invention comprises at least one silicone of formula (I) below:

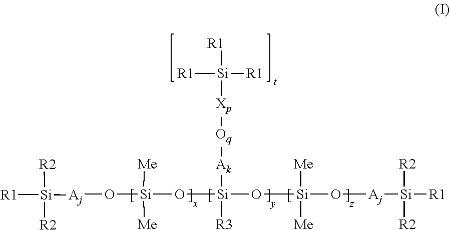

wherein:
R1 independently represents a hydroxyl group or an alkoxy group containing from 1 to 2 carbon atoms or an alkyl group containing from 1 to 10 carbon atoms, preferably from 1 to 4 carbon atoms;
R2 independently represents an alkyl group containing from 1 to 10 carbon atoms, preferably from 1 to 4 carbon atoms, or an alkoxy group containing from 1 to 2 carbon atoms or a hydroxyl group or a monovalent radical of formula —C_{q}H_{2q}L wherein q is a number ranging from 2 to 8, and L is an optionally quaternized amino group chosen from the groups:

—N(R″)₂;—N+(R″)₃A⁻;—NR″-Q-N(R″)₂ and —NR″-Q-N+(R″)₃A⁻,

wherein R″, which may be identical or different, represents a hydrogen atom, a phenyl group, a benzyl group or a saturated monovalent hydrocarbon-based radical, for example a C₁-C₂₀ alkyl group; Q denotes a linear or branched group of formula CᵣH₂ᵣ, r being an integer ranging from 2 to 6, preferably from 2 to 4; and A⁻ represents a cosmetically acceptable anion, notably a halide such as fluoride, chloride, bromide or iodide;
R3 represents a hydroxyl group; an alkyl group containing from 1 to 10 carbon atoms, notably from 1 to 4 carbon atoms, said alkyl group being optionally substituted with at least one group chosen from a hydroxyl group (OH) or a thiol group (SH); a cycloalkyl group containing from 3 to 20 carbon atoms, notably from 5 to 6 carbon atoms, said cycloalkyl being optionally substituted with at least one group chosen from a hydroxyl group (OH) or a thiol group (SH); an alkoxy group containing from 1 to 2 carbon atoms, optionally substituted with at least one group chosen from a hydroxyl group (OH) or a thiol group (SH); an aryl group containing from 6 to 12 carbon atoms, optionally substituted with at least one group chosen from a hydroxyl group (OH) or a thiol group (SH); or a radical —(X)_{p′},—Si(R2)₃ or a monovalent radical of formula —C_{q}H_{2q}L wherein q is a number ranging from 2 to 8, and L is an amino group as described previously;
A represents a saturated divalent hydrocarbon-based radical containing 1 carbon atom;
X represents a hydrogen atom or a saturated, monovalent or divalent hydrocarbon-based radical, containing 1 carbon atom;
p is an integer ranging from 0 to 6; p′ is an integer equal to 0 or 1 or 2 or 3;
k is an integer ranging from 0 to 6;
q denotes an integer equal to 0 or 1;
j denotes an integer equal to 0 or 1 or 2;
t denotes an integer equal to 0 or 1;
y denotes an integer ranging from 0 to 10, z denotes an integer ranging from 0 to 500 with x+z ranging from 0 to 500 and x+y+z≥4;
   it being understood that if X represents a hydrogen atom, then t=0 and p=1 and if p=0, then t=1 and q=1 and that at least one of the radicals R1 or R3 denotes a hydroxyl radical or an alkoxy radical containing from 1 to 2 carbon atoms or a radical —(X)_{p′},—Si(R2)₃ or a monovalent radical of formula —C_{q}H_{2q}L wherein q is a number ranging from 2 to 8, and L is an amino group as described previously.

Preferably, the silicone(s) of formula (I) are such that:
R1 independently represents a hydroxyl group or an alkoxy group containing from 1 to 2 carbon atoms such as methoxy or ethoxy, or an alkyl group containing from 1 to 2 carbon atoms such as a methyl;
R2 independently represents an alkyl group containing from 1 to 4 carbon atoms, more preferentially 1 to 2 carbon atoms such as a methyl or an alkoxy group containing from 1 to 2 carbon atoms or a hydroxyl group;
R3 represents a hydroxyl group or an alkyl group containing from 1 to 10 carbon atoms, notably from 1 to 4 carbon atoms, or an alkoxy group containing from 1 to 2 carbon atoms or a radical —(X)_{p′},—Si(R2)₃ or a monovalent radical of formula —C_{q}H_{2q}L in which q is a number ranging from 2 to 8, and L is an amino group chosen from the groups:

—N(R4)₂;

—N(R4)—CH₂—CH₂—N(R4)₂;

wherein R4 represents a hydrogen atom; a phenyl group; a benzyl group or a saturated monovalent hydrocarbon-based radical, for example a C₁-C₂₀ alkyl group;
A represents a saturated divalent hydrocarbon-based radical containing 1 carbon atom;
X represents a hydrogen atom or a saturated, monovalent or divalent hydrocarbon-based radical, containing 1 carbon atom;
p denotes an integer ranging from 0 to 6, and p′ is an integer equal to 0 or 1 or 2 or 3,
j is equal to 0;
k is an integer equal to 0 or 1;
q denotes an integer equal to 0 or 1;
t denotes an integer equal to 0 or 1; and
y denotes an integer ranging from 0 to 10, z denotes an integer ranging from 0 to 500 with x+z ranging from 0 to 500 and x+y+z≥4; and
   it being understood that if X represents a hydrogen atom, then t=0 and p=1 and that if p=0, then t=1 and q=1 and that at least one of the radicals R1 or R3 denotes a hydroxyl radical or an alkoxy radical containing from 1 to 2 carbon atoms or a monovalent radical of formula —C_{q}H_{2q}L as described previously.

More preferentially, the silicone(s) of formula (I) are such that:
R1 independently represents a hydroxyl group or an alkoxy group containing from 1 to 2 carbon atoms such as methoxy or ethoxy, or an alkyl group containing from 1 to 2 carbon atoms such as methyl;
R2 independently represents an alkyl group containing from 1 to 2 carbon atoms such as a methyl or an alkoxy group containing from 1 to 2 carbon atoms or a hydroxyl group;
R3 represents a monovalent radical of formula —C_{q}H_{2q}L wherein q is a number ranging from 2 to 8 and L is an amino group chosen from the groups:

—N(R4)₂;

N(R4)—CH₂—CH₂—N(R4)₂;

wherein R4 represents a hydrogen atom; a phenyl group; a benzyl group or a saturated monovalent hydrocarbon-based radical, for example a C₁-C₂₀ alkyl group;
A represents a saturated divalent hydrocarbon-based radical containing 1 carbon atom;
X represents a hydrogen atom, and p denotes an integer equal to 1,
j is equal to 0;
k is equal to 1;
q denotes an integer equal to 0;
t denotes an integer equal to 0;
y denotes an integer ranging from 0 to 10, z denotes an integer ranging from 0 to 500 with x+z ranging from 0 to 500 and x+y+z≥4;
   it being understood that at least one of the radicals R1 or R3 denotes a hydroxyl radical or an alkoxy radical containing from 1 to 2 carbon atoms or a monovalent radical of formula —C_{q}H_{2q}L as described previously.

The silicone(s) of formula (I) used in the context of the invention may be chosen from:
the compounds of formula (Ia) below:

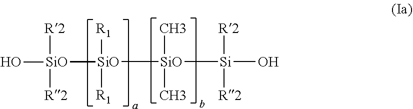

in which:
R₁, which may be identical or different, independently represents a hydroxyl group; an alkyl group containing from 1 to 4 carbon atoms, preferably from 1 to 2 carbon atoms such as a methyl, or an alkoxy group containing from 1 to 2 carbon atoms;
R′2 and R″2, which may be identical or different, independently represent an alkyl group containing from 1 to 4 carbon atoms, notably from 1 to 2 carbon atoms such as a methyl, or a hydroxyl group;
a denotes an integer ranging from 0 to 10, b denotes an integer ranging from 0 to 500 with a+b≥4.
the compounds of formula (Ib) below:

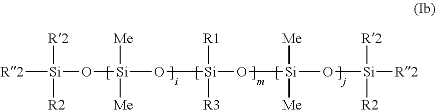

wherein:
R1 independently represents an alkyl group containing from 1 to 4 carbon atoms, and very particularly a methyl; or an alkoxy group containing from 1 to 2 carbon atoms;
R2, R′2 and R″2, which may be identical or different, independently represent an alkyl group containing from 1 to 4 carbon atoms and in particular from 1 to 2 carbon atoms, or a hydroxyl group or an alkoxy group containing from 1 to 2 carbon atoms;
R3 represents a group —(X)_{p′},—Si(OR4)₃ with R4 representing an alkyl group containing from 1 to 2 carbon atoms, X representing a saturated divalent hydrocarbon-based radical containing 1 carbon atom, and p′ denoting an integer ranging from 1 to 3;
m denotes an integer ranging from 0 to 3;
i denotes an integer ranging from 0 to 10, j denotes an integer ranging from 0 to 500, i+j ranging from 0 to 510 with i+j≥4;
the compounds of formula (Ic) below:

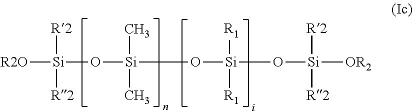

wherein:
R1, which may be identical or different, represents an alkyl group containing from 1 to 4 carbon atoms, preferably from 1 to 2 carbon atoms, such as a methyl; or an alkoxy group containing from 1 to 2 carbon atoms;
R₂ independently represents an alkyl group containing from 1 to 2 carbon atoms such as a methyl or ethyl;
R′2 and R″2, which may be identical or different, independently represent an alkyl group containing from 1 to 4 carbon atoms, more preferentially from 1 to 2 carbon atoms, or an alkoxy group containing from 1 to 2 carbon atoms such as a methoxy or ethoxy;
i denotes an integer ranging from 0 to 10, n denotes an integer ranging from 0 to 500 with n+i ranging from 0 to 510 with n+i≥4;
the compounds of formula (Id):

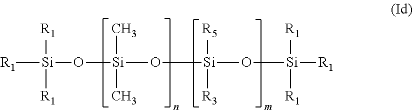

wherein:
m and n are numbers such that the sum (m+n) ranges from 1 to 500, n denoting a number ranging from 0 to 500, and m denoting a number ranging from 1 to 500;
R₁, which may be identical or different, independently represents an alkyl group containing from 1 to 4 carbon atoms, more preferentially from 1 to 2 carbon atoms, for example a methyl; or a hydroxyl group or an alkoxy group containing from 1 to 2 carbon atoms;
R₃ represents a monovalent radical of formula —C_{q}H_{2q}L wherein q is a number ranging from 2 to 8 and L is an amino group chosen from the following groups:

—N(R₄)₂;

N(R₄)—CH₂—CH₂—N(R₄)₂;

wherein R₄ represents a hydrogen atom; a phenyl group; a benzyl group or a saturated monovalent hydrocarbon-based radical, for example a C₁-C₂₀ alkyl group; and
R₅ represents a hydroxyl group or an alkyl group containing from 1 to 2 carbon atoms, for example a methyl, or an alkoxy group containing from 1 to 2 carbon atoms;
and mixtures thereof.

Among the silicones of formula (Ia), mention may be made of polydimethylsiloxanes (PDMS) bearing hydroxyl end functions, such as the compounds sold by the company Shin-Etsu under the name KF-9701 or X-21-5841, or those sold by the company Sigma-Aldrich under the reference 481939 (Mn ˜550, ˜25 cSt), 481955 (˜65 cSt), or 481963 (˜750 cSt).

Mention may also be made of the compounds sold by the company Gelest under the name DMS-S12 (16-32 cSt), DMS-S15 (45-85 cSt), DMS-S21 (90-120 cSt), DMS-S27 (700-800 cSt) or DMS-S31 (˜1000 cSt).

Among the silicones of formula (Ib), mention may be made of polydimethylsiloxanes (PDMS) bearing trialkoxysilane side functions, such as those sold by the company Siltech under the name Silmer TMS C50.

Among the silicones of formula (Ic), mention may be made of polydimethylsiloxanes (PDMS) bearing trialkoxysilane end functions, such as those sold by the company Power Chemical under the name SiSiB® PF2110, or those sold by the company Siltech under the name Silmer TMS Di-10 or Silmer TMS Di-50.

Among the silicones of formula (Id), mention may be made of the compound having the trade name KF-857, sold by the company Shin-Etsu, or the compound having the trade name KF-862, sold by the company Shin-Etsu.

Preferably, the silicone(s) of formula (I) are chosen from the silicones of formulae (Ia) and (Id) and mixtures thereof.

More preferentially, the silicone(s) of formula (I) used in the context of the invention are chosen from the compounds of formula (Ia) in which:
R₁ independently represents an alkyl group containing from 1 to 4 carbon atoms, preferably from 1 to 2 carbon atoms such as a methyl;
R′2 and R″2 independently represent an alkyl group containing from 1 to 4 carbon atoms, more particularly from 1 to 2 carbon atoms such as methyl;
a denotes an integer ranging from 0 to 10, b denotes an integer ranging from 0 to 500 with a+b≥4.

Even more preferentially, the silicone(s) of formula (I) are represented by the formula (Ia) below:

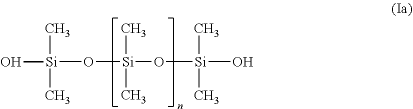

wherein n denotes an integer ranging from 4 to 500.

Preferably, the silicone(s) of formula (I) have a weight-average molecular mass ranging from 460 to 5000 g/mol, more preferentially from 500 to 5000 g/mol. The weight-average molecular masses of these silicones may be measured by gel permeation chromatography (GPC) at ambient temperature (25° C.), as polystyrene equivalent. The columns used are μ styragel columns. The eluent is THE and the flow rate is 1 ml/min. 200 μl of a 0.5% by weight solution of silicone in THE are injected. Detection is performed by refractometry and UV-metry.

Preferably, the silicone(s) of formula (I) is (are) non-cyclic.

The expression “non-cyclic silicone” is understood to mean a silicone, the silicone chain(s) of which do not form a ring.

The silicone(s) of formula (I) may be present in a total amount ranging from 0.1% to 30% by weight, preferably from 1% to 20% by weight and more preferentially from 1% to 15% by weight relative to the total weight of the composition.

### Silicone of Formula (II):

The composition according to the invention comprises at least one silicone of formula (II) below:

R¹ₐR²_{b}R³_{c}Si O_{(4-a-b-c)/2} (II)

wherein:
R¹ represents an alkyl group containing from 1 to 30 carbon atoms, a cycloalkyl group containing from 5 to 10 carbon atoms, an aryl group containing from 6 to 10 carbon atoms, an aralkyl group containing from 7 to 14 carbon atoms, an alkyl group containing from 3 to 10 carbon atoms substituted with at least one fluorine atom or an organic group of formula (A) below:

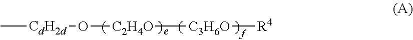

wherein:
R₄ represents an alkyl group containing from 1 to 30 carbon atoms or an acyl group containing from 1 to 30 carbon atoms,
d denotes an integer ranging from 0 to 15,
e denotes an integer ranging from 0 to 50, and
f denotes an integer ranging from 0 to 50;
R² represents a hydroxyl group or an alkoxy group containing from 1 to 6 carbon atoms, or a group of formula (B) below:

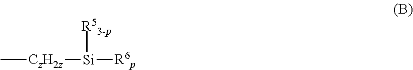

wherein:
R⁵ represents an alkyl group containing from 1 to 6 carbon atoms or a cycloalkyl group containing from 5 to 6 carbon atoms or a phenyl group,
R⁶ represents an alkoxy group containing from 1 to 6 carbon atoms,
z denotes an integer ranging from 2 to 8, and
p denotes an integer ranging from 1 to 3;
R³ represents a silicone compound of formula (C) below:

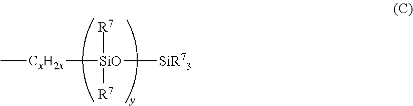

wherein:
R⁷ independently represents an alkyl group containing from 1 to 30 carbon atoms, a cycloalkyl group containing from 5 to 10 carbon atoms, an aryl group containing from 6 to 10 carbon atoms, an aralkyl group containing from 7 to 14 carbon atoms or an alkyl group containing from 3 to 10 carbon atoms substituted with at least one fluorine atom,
x denotes an integer ranging from 1 to 5; and
y denotes an integer ranging from 0 to 500;
a denotes an integer ranging from 1.0 to 2.5,
b denotes an integer ranging from 0.001 to 1.5, and
c denotes an integer ranging from 0 to 1.5;
   it being understood that when a plurality of R¹ to R⁷ radicals are present, the R¹ to R⁷ radicals are identical to or different from one another.

Preferably, the silicone(s) of formula (II) are such that:
R¹ represents an alkyl group containing from 1 to 30 carbon atoms, a cycloalkyl group containing from 5 to 10 carbon atoms, an aryl group containing from 6 to 10 carbon atoms, an aralkyl group containing from 7 to 14 carbon atoms or an alkyl group containing from 3 to 10 carbon atoms substituted with at least one fluorine atom,
R² represents a hydroxyl group or an alkoxy group containing from 1 to 6 carbon atoms or a group of formula (B) wherein R⁶ represents an alkoxy group containing from 1 to 6 carbon atoms, preferably from 1 to 3 carbon atoms, z denotes an integer equal to 2, and p denotes an integer equal to 3;
R³ represents a silicone compound of formula (C) wherein R⁷ independently represents an alkyl group containing from 1 to 30 carbon atoms, x denotes an integer ranging from 1 to 5, and y denotes an integer ranging from 0 to 500,
a denotes an integer ranging from 1.0 to 2.5,
b denotes an integer ranging from 0.001 to 1.5, and
c denotes an integer ranging from 0 to 1.5.

Among the silicone compounds of formula (II), mention may be made of the compound having the trade name KF-9908, sold by the company Shin-Etsu (INCI name: Triethoxysilylethyl Polydimethylsiloxyethyl Dimethicone).

Preferably, the silicone(s) of formula (II) have a weight-average molecular mass ranging from 300 to 100 000 g/mol, more preferentially from 1000 to 10 000 g/mol.

The silicone(s) of formula (II) may be present in a total amount ranging from 0.1% to 30% by weight, preferably from 1% to 25% by weight and more preferentially from 1% to 20% by weight, better still from 3% to 15% by weight relative to the total weight of the composition.

### Alkoxysilane:

The composition according to the invention may comprise at least one alkoxysilane chosen from the compounds of formula (III) below, oligomers thereof and/or mixtures thereof:

R¹ₓSi(OR²)₍₄₋ₓ₎ (III)

wherein:
R¹ represents an alkoxy group containing from 1 to 10 carbon atoms, an amino group NH₂, a linear or branched, saturated or unsaturated, cyclic or acyclic C₁ to C₂₂, notably C₁ to C₂₀, hydrocarbon-based radical which may be substituted with at least one group chosen from a hydroxyl group (OH); a thiol group (SH); an alkoxy group containing from 1 to 10 carbon atoms; an aryl group containing from 6 to 30 carbon atoms; an amino group NH₂; a (di)alkylamino group NR³R⁴ wherein R³ and R⁴ independently denote a hydrogen atom, a linear or branched alkyl group containing from 1 to 20 carbon atoms, an aminoalkyl group containing from 1 to 20 carbon atoms, an aryl group containing from 6 to 12 carbon atoms or a linear or branched (cyclo)alkyl group containing from 1 to 20 carbon atoms, notably from 1 to 10 carbon atoms; it being possible for R¹ to be interrupted with at least one heteroatom chosen from O, S, NH, NR³, a carbonyl group (CO) and combinations thereof,
R² represents a hydrogen atom or an alkyl group containing from 1 to 20 carbon atoms, preferably from 1 to 6 carbon atoms;
x denotes an integer ranging from 1 to 3;
   it being understood that if all the R² radicals represent a hydrogen atom, then R¹ represents an alkoxy group having from 1 to 10 carbon atoms.

R¹ and R² may be identical or different.

The term “oligomer” means compound(s) including at least two silicon atoms, obtained by oligomerization or polymerization of the compounds of formula (III).

Preferably, R¹ represents an alkoxy group containing from 1 to 10 carbon atoms, such as an ethoxy, or R¹ represents a linear or branched, saturated C₁ to C₂₂, notably C₁ to C₂₀ hydrocarbon-based radical, which is preferably linear, said hydrocarbon-based radical possibly being substituted with at least one amino group NH₂ or alkylamino group NHR, in which R denotes a linear or branched alkyl radical containing from 1 to 20 carbon atoms, notably from 1 to 10 carbon atoms, it being possible for R¹ to be interrupted with at least one heteroatom chosen from O, S, NH, NR³, a carbonyl group (CO) and combinations thereof.

More preferentially, R¹ represents a saturated linear C₁ to C₆ hydrocarbon-based radical, which may be substituted with an amino group NH₂.

Preferably, R² represents an alkyl group containing from 1 to 10 carbon atoms, preferably from 1 to 6 carbon atoms, more preferentially an ethyl.

The alkoxysilane(s) of formula (III), the oligomers thereof and/or mixtures thereof may be chosen from:
the compounds of formula (IIIa) and/or (IIIb) below, oligomers thereof, alone or as a mixture:

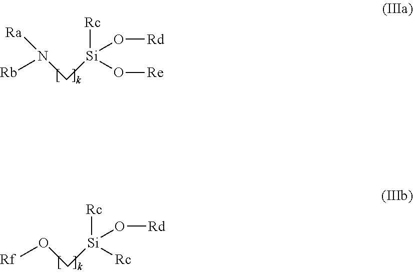

wherein:
Ra and Rb, which may be identical or different, represent a hydrogen atom; an alkyl group containing from 1 to 20 carbon atoms, preferably from 1 to 10 carbon atoms and notably from 1 to 4 carbon atoms; a cycloalkyl group containing from 3 to 20 carbon atoms; an aryl group containing from 6 to 12 carbon atoms; an aminoalkyl group containing from 1 to 20 carbon atoms;
Rc represents an alkyl group containing from 1 to 20 carbon atoms, preferably from 1 to 10 carbon atoms, more preferentially from 1 to 4 carbon atoms and in particular from 1 to 2 carbon atoms such as a methyl, it being possible for said alkyl group to be substituted with an aryl group; an alkoxy group containing from 1 to 10 carbon atoms, preferably from 1 to 4 carbon atoms and in particular from 1 to 2 carbon atoms such as an ethoxy, or an aryl group containing from 6 to 12 carbon atoms;
Rd and Re, which may be identical or different, represent an alkyl group containing from 1 to 10 carbon atoms, preferably from 1 to 4 carbon atoms, in particular from 1 to 2 carbon atoms, such as an ethyl;
k denotes an integer ranging from 0 to 5, preferably ranging from 0 to 3;
Rf represents a hydrogen atom; an alkyl group containing from 1 to 10 carbon atoms and notably from 1 to 4 carbon atoms; or a group of formula (IV) below:

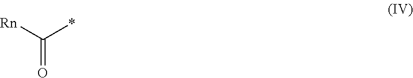

wherein Rn represents a hydroxyl group (OH); an alkyl group containing from 1 to 10 carbon atoms, preferably a methyl.

Among the alkoxysilanes of formula (IIIa), the oligomers thereof and/or mixtures thereof, mention may notably be made of 3-aminopropyltriethoxysilane (APTES), 3-aminopropylmethyldiethoxysilane (APMDES) and N-cyclohexylaminomethyltriethoxysilane.

APTES may be purchased, for example, from the company Dow Corning under the name Xiameter OFS-6011 Silane or from the company Momentive Performance Materials under the name Silsoft A-1100 or from the company Shin-Etsu under the name KBE-903.

The compounds of formula (IIIa) may also denote Dynasylan SIVO 210 or Dynasylan 1505 sold by the company Evonik.

N-Cyclohexylaminomethyltriethoxysilane may be purchased, for example, from the company Wacker under the name Geniosil XL 926.

Among the alkoxysilanes of formula (IIIb), the oligomers thereof and/or mixtures thereof, mention may notably be made of tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof. TEOS may be purchased, for example, from the company Evonik under the name Dynasylan® A or Dynasylan® A SQ. MTES may be purchased, for example, from the company Evonik under the name Dynasylan® MTES. DMDES may be purchased, for example, from the company Gelest under the reference SID3404.0.

Preferably, the alkoxysilane(s) chosen from the compounds of formula (III), the oligomers thereof and/or mixtures thereof are chosen from 3-aminopropyltriethoxysilane (APTES), 3-aminopropylmethyldiethoxysilane (APMDES), N-cyclohexylaminomethyltriethoxysilane, tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof, more preferentially 3-aminopropyltriethoxysilane (APTES), tetraethoxysilane (TEOS) and mixtures thereof.

Preferably, the alkoxysilane(s) of formula (III), the oligomers thereof and/or mixtures thereof are chosen from the compounds of formula (IIIa) below:

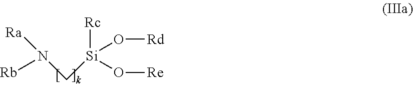

wherein:
Ra and Rb, which are identical, represent a hydrogen atom or Ra denotes a hydrogen atom and Rb denotes a C₅-C₆ cycloalkyl radical such as cyclohexyl;
Rc represents an alkyl group containing from 1 to 10 carbon atoms, notably from 1 to 4 carbon atoms and in particular from 1 to 2 carbon atoms, preferably a methyl, or an alkoxy group containing from 1 to 4 carbon atoms, preferably from 1 to 2 carbon atoms, preferably an ethoxy;
Rd and Re, which may be identical or different, represent an alkyl group containing from 1 to 10 carbon atoms, preferably from 1 to 4 carbon atoms, such as an ethyl;
k denotes an integer ranging from 1 to 3 and more particularly 1 or 3.

Preferentially, the compounds of formula (IIIa) are such that Ra and Rb represent a hydrogen atom, Rc represents an ethoxy group, Rd and Re are identical and represent an ethyl and k is equal to 3.

Preferably, the alkoxysilane(s) of formula (V) are chosen from 3-aminopropyltriethoxysilane (APTES), 3-aminopropylmethyldiethoxysilane (APMDES), N-cyclohexylaminomethyltriethoxysilane and mixtures thereof, more preferentially 3-aminopropyltriethoxysilane (APTES).

According to another preferred embodiment, the alkoxysilane(s) of formula (III), the oligomers thereof and/or mixtures thereof are chosen from the compounds of formula (IIIb) below:

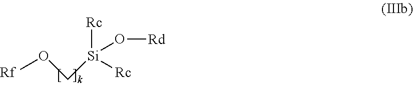

wherein:
Rc represents an alkoxy group containing from 1 to 10 carbon atoms, preferably from 1 to 4 carbon atoms and in particular from 1 to 2 carbon atoms, such as an ethoxy; an aryl group containing from 6 to 12 carbon atoms or an alkyl group containing from 1 to 10 carbon atoms substituted with an aryl group, preferably 1 to 2 carbon atoms substituted with an aryl group;
Rd represents an alkyl group containing from 1 to 10 carbon atoms, preferably from 1 to 4 carbon atoms, in particular from 1 to 2 carbon atoms, such as an ethyl;
k denotes an integer ranging from 0 to 3, preferably equal to 0;
Rf represents a hydrogen atom or an alkyl group containing from 1 to 10 carbon atoms and notably from 1 to 4 carbon atoms such as an ethyl.

According to another more preferred embodiment, the alkoxysilane of formula (III), the oligomers thereof and/or mixtures thereof, is tetraethoxysilane (TEOS).

The alkoxysilane(s) of formula (III), oligomers thereof and/or mixtures thereof may be present in a total amount ranging from 0.01% to 10% by weight, preferably from 0.05% to 5% by weight and better still from 0.07% to 3% by weight, relative to the total weight of the composition.

### Weight Ratio:

The composition according to the invention may comprise a silicone of formula (I)/silicone of formula (II) weight ratio ranging from 95:5 to 5:95.

Preferably, the silicone of formula (I)/silicone of formula (II) weight ratio varies from 90:10 to 10:90, preferentially from 80:20 to 20:80 and more preferentially from 70:30 to 30:70.

According to one particular embodiment, the silicone of formula (Ia)/silicone of formula (II) weight ratio varies from 90:10 to 10:90, preferentially from 80:20 to 20:80 and more preferentially from 70:30 to 30:70.

### Coloring Agent:

The composition according to the invention comprises at least one coloring agent chosen from pigments, direct dyes and mixtures thereof.

Preferably, the composition according to the invention comprises one or more pigments.

Preferably, the coloring agent(s) are chosen from pigments.

The term “pigment” is understood to mean any pigment that gives color to keratin materials. Their solubility in water at 25° C. and at atmospheric pressure (760 mmHg) is less than 0.05% by weight, and preferably less than 0.01% by weight.

The pigments which can be used are in particular chosen from the organic and/or inorganic pigments known in the art, in particular those which are described in the Kirk-Othmer Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from inorganic pigments, organic pigments, lakes, special effect pigments such as pearlescent agents or glitter flakes, and mixtures thereof.

The pigment may be an inorganic pigment. The term “inorganic pigment” is understood to mean any pigment which satisfies the definition in Ullmann's Encyclopedia in the chapter “Pigments, Inorganic”. Mention may be made, among the inorganic pigments of use in the present invention, of iron oxides, chromium oxides, manganese violet, ultramarine blue, chromium hydrate, ferric blue and titanium oxide.

The pigment may be an organic pigment. The term “organic pigment” is understood to mean any pigment that satisfies the definition in Ullmann's Encyclopedia in the chapter “Pigments, Organic”.

The organic pigment can in particular be chosen from nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or colored organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771. Examples that may also be mentioned include pigment pastes of organic pigments, such as the products sold by the company Hoechst under the names:
Cosmenyl Yellow 10G: Pigment Yellow 3 (CI 11710);
Cosmenyl Yellow G: Pigment Yellow 1 (CI 11680);
Cosmenyl Orange GR: Pigment Orange 43 (CI 71105);
Cosmenyl Red R: Pigment Red 4 (CI 12085);
Cosmenyl Carmine FB: Pigment Red 5 (CI 12490);
Cosmenyl Violet RL: Pigment Violet 23 (CI 51319);
Cosmenyl Blue A2R: Pigment Blue 15.1 (CI 74160);
Cosmenyl Green GG: Pigment Green 7 (CI 74260);
Cosmenyl Black R: Pigment Black 7 (CI 77266).

The pigments in accordance with the invention may also be in the form of composite pigments, as described in patent EP 1 184 426. These composite pigments may be composed notably of particles including an inorganic core, at least one binder for attaching the organic pigments to the core, and at least one organic pigment which at least partially covers the core.

The organic pigment may also be a lake. The term “lake” is intended to mean dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate and aluminum. Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a special effect pigment. The term “special effect pigments” means pigments that generally create a colored appearance (characterized by a certain shade, a certain vivacity and a certain level of luminance) that is non-uniform and that changes as a function of the conditions of observation (light, temperature, angles of observation, etc.). They thereby differ from colored pigments, which afford a standard uniform opaque, semi-transparent or transparent shade.

Several types of special effect pigments exist: those with a low refractive index, such as fluorescent or photochromic pigments, and those with a higher refractive index, such as pearlescent agents, interference pigments or glitter flakes.

Examples of special effect pigments that may be mentioned include pearlescent pigments such as mica covered with titanium or with bismuth oxychloride, colored pearlescent pigments such as mica covered with titanium and with iron oxides, mica covered with iron oxide, mica covered with titanium and notably with ferric blue or with chromium oxide, mica covered with titanium and with an organic pigment as defined previously, and also pearlescent pigments based on bismuth oxychloride. Mention may be made, as pearlescent pigments, of the pearlescent agents Cellini sold by BASF (mica-TiO₂-lake), Prestige sold by Eckart (mica-TiO₂), Prestige Bronze sold by Eckart (mica-Fe₂O₃) or Colorona sold by Merck (mica-TiO₂—Fe₂O₃).

Mention may also be made of the gold-colored pearlescent agents sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze pearlescent agents sold notably by the company Merck under the name Bronze fine (17384) (Colorona) and Bronze (17353) (Colorona) and by the company BASF under the name Super bronze (Cloisonne); the orange pearlescent agents sold notably by the company BASF under the name Orange 363C (Cloisonne) and Orange MCR 101 (Cosmica) and by the company Merck under the name Passion orange (Colorona) and Matte orange (17449) (Microna); the brown pearlescent agents sold notably by the company BASF under the name Nu-antique copper 340XB (Cloisonne) and Brown CL4509 (Chromalite); the pearlescent agents with a copper glint sold notably by the company BASF under the name Copper 340A (Timica); the pearlescent agents with a red glint sold notably by the company Merck under the name Sienna fine (17386) (Colorona); the pearlescent agents with a yellow glint sold notably by the company BASF under the name Yellow (4502) (Chromalite); the red pearlescent agents with a gold glint sold notably by the company BASF under the name Sunstone G012 (Gemtone); the pink pearlescent agents sold notably by the company BASF under the name Tan opal G005 (Gemtone); the black pearlescent agents with a gold glint sold notably by the company BASF under the name Nu antique bronze 240 AB (Timica), the blue pearlescent agents sold notably by the company Merck under the name Matte blue (17433) (Microna), the white pearlescent agents with a silvery glint sold notably by the company Merck under the name Xirona Silver, and the golden-green pink-orange pearlescent agents sold notably by the company Merck under the name Indian summer (Xirona), and mixtures thereof.

Still as examples of pearlescent agents, mention may also be made of particles including a borosilicate substrate coated with titanium oxide.

Particles comprising a glass substrate coated with titanium oxide are notably sold under the name Metashine MC1080RY by the company Toyal.

Finally, examples of pearlescent agents that may also be mentioned include polyethylene terephthalate glitter flakes, notably those sold by Meadowbrook Inventions under the name Silver 1P 0.004X0.004 (silvery glitter flakes). It is also possible to envisage multilayer pigments based on synthetic substrates, such as alumina, silica, calcium sodium borosilicate, calcium aluminum borosilicate and aluminum. The special effect pigments may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. brighter points that contrast with their environment, making them appear to sparkle. The reflective particles may be selected so as not to significantly alter the coloring effect generated by the coloring agents with which they are combined, and more particularly so as to optimize this effect in terms of color rendition. They may more particularly have a yellow, pink, red, bronze, orange, brown, gold and/or coppery color or glint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically. When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or inorganic materials. More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in documents JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminum, iron, chromium, nickel, molybdenum, gold, copper, zinc, tin, magnesium, steel, bronze or titanium, said substrate being coated with at least one layer of at least one metal oxide, such as titanium oxide, aluminum oxide, iron oxide, cerium oxide, chromium oxide, silicon oxides and mixtures thereof.

Examples that may be mentioned include aluminum powder, bronze powder or copper powder coated with SiO₂ sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Special effect pigments also comprise fluorescent pigments, whether these are substances which are fluorescent in daylight or which produce an ultraviolet fluorescence, phosphorescent pigments, photochromic pigments, thermochromic pigments and quantum dots, for example sold by Quantum Dots Corporation.

The variety of the pigments that may be used in the present invention makes it possible to obtain a rich palette of colors and also specific optical effects, such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by Avecia, polyglyceryl-2 dipolyhydroxystearate (CTFA name) sold under the reference Dehymyls PGPH by Henkel, or else polyhydroxystearic acid such as the product sold under the reference Arlacel P100 by Uniqema, and mixtures thereof.

As other dispersants that may be used in the compositions of the invention, mention may be made of quaternary ammonium derivatives of polycondensed fatty acids, for instance Solsperse 17 000 sold by Avecia, and polydimethylsiloxane/oxypropylene mixtures such as those sold by Dow Corning under the references DC2-5185 and DC2-5225 C.

The pigments used in the composition may be surface-treated with an organic agent. Thus, the pigments that have been surface-treated beforehand, which are useful in the context of the invention, are pigments that have totally or partially undergone a surface treatment of chemical, electronic, electrochemical, mechanochemical or mechanical nature, with an organic agent such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof, anionic surfactants; lecithins; sodium, potassium, magnesium, iron, titanium, zinc or aluminum salts of fatty acids, for example aluminum stearate or laurate; metal alkoxides; polyethylene; (meth)acrylic polymers, for example polymethyl methacrylates; polymers and copolymers containing acrylate units; alkanolamines; silicone compounds, for example silicones, in particular polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment can thus be carried out, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used. The agent for the surface treatment may represent from 0.1% to 50% by weight of the total weight of the surface-treated pigment, preferably from 0.5% to 30% by weight and even more preferentially from 1% to 20% by weight of the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethylsiloxysilicate treatment, for instance the Covasil 4.05 surface treatment sold by LCW;
a magnesium myristate treatment, for instance the MM surface treatment sold by LCW;
an aluminum dimyristate treatment, such as the MI surface treatment sold by Miyoshi;
a perfluoropolymethyl isopropyl ether treatment, for instance the FHC surface treatment sold by LCW;
an isostearyl sebacate treatment, such as the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or inorganic pigments in submicron-sized particulate form in the dye composition.

The term “submicron” or “submicronic” refers to pigments having a particle size that has been micronized by a micronization method and having a mean particle size of less than a micrometer (μm), in particular between 0.1 and 0.9 μm, and preferably between 0.2 and 0.6 μm.

According to one embodiment, the dispersant and the pigment(s) are present in a (dispersant:pigment) amount of between 1:4 and 4:1, particularly between 1.5:3.5 and 3.5:1 or better still between 1.75:3 and 3:1.

The dispersant(s) may thus have a silicone backbone, such as silicone polyether and dispersants of aminosilicone type other than the silicones of formula (I) or alkoxysilanes described previously. Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of aminosilicone type other than the silicones of formula (I) or alkoxysilanes described previously and are cationic.

Preferably, the pigment(s) is (are) chosen from mineral, mixed mineral-organic or organic pigments.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

The composition according to the invention can comprise one or more direct dye(s).

The term “direct dye” means natural and/or synthetic dyes, other than oxidation dyes.

These are dyes that will spread superficially on the fiber.

They can be ionic or nonionic, preferably anionic, cationic or nonionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (V) and (VI) and the azo cationic dyes (VII) and (VIII) below:

Het⁺—C(Ra)═N—N(Rb)—Ar,Q (V)

Het⁺—N(Ra)—N═C(Rb)—Ar,Q- (VI)

Het⁺—N═N—Ar,Q- (VII)

Ar⁺—N═N—Ar″,Q- (VIII)

### Formulae (V) to (VIII) Wherein:

Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₅)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group; or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (V) to (VIII) as defined previously. More particularly, the cationic direct dyes bearing an endocyclic cationic charge described in patent applications WO 95/15144, WO 95/01772 and EP 714 954.

Preferentially the following direct dyes:

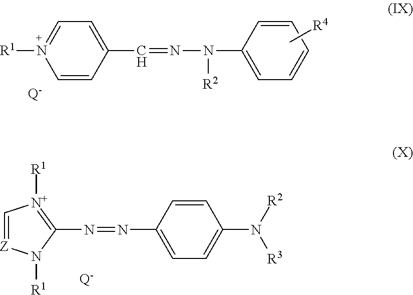

formulae (IX) and (X) wherein:
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q⁻ is an anionic counterion as defined previously, in particular a halide, such as chloride, or an alkyl sulfate, such as methyl sulfate or mesityl.

In particular, the dyes of formulae (IX) and (X) are chosen from Basic Red 51, Basic Yellow 87 and Basic Orange 31 or derivatives thereof with Q⁻ being an anionic counterion as defined previously, particularly a halide such as chloride, or an alkyl sulfate such as methyl sulfate or mesityl.

The direct dyes can be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

Mention may be made, as acid dyes according to the invention, of the dyes of formulae (XI), (XI′), (XII), (XII′), (XIII), (XIII′), (XIV), (XIV′), (XV), (XVI), (XVII) and (XVIII) below:

a) the diaryl anionic azo dyes of formula (XI) or (XI′):

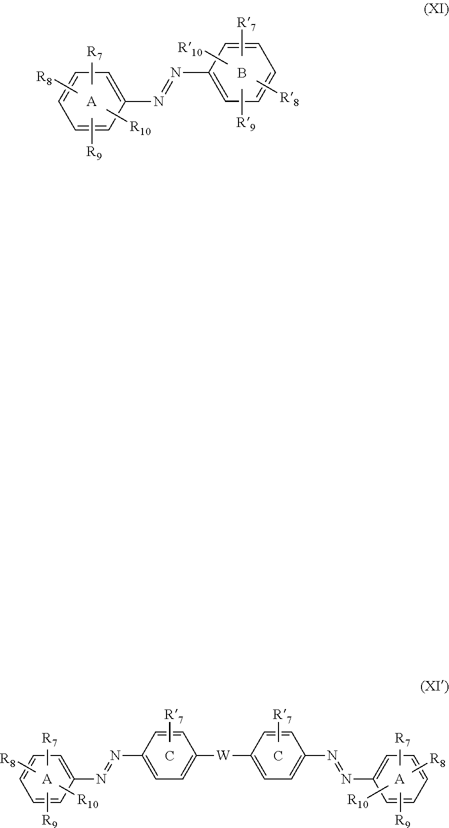

formulae (XI) and (XI′) wherein:
R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
R″—S(O)₂—, with R″ representing a hydrogen atom or an alkyl, aryl, (di)(alkyl)amino or aryl(alkyl)amino group; preferentially a phenylamino or phenyl group;
R′″—S(O)₂—X′— with R′″ representing an optionally substituted alkyl or aryl group, X′ as defined previously;
(di)(alkyl)amino;
aryl(alkyl)amino optionally substituted with one or more groups chosen from i) nitro;
   ii) nitroso; iii) (O)₂S(O⁻)—, M+ and iv) alkoxy with M⁺ as defined previously;
optionally substituted heteroaryl; preferentially a benzothiazolyl group;
cycloalkyl; notably cyclohexyl;
Ar—N═N— with Ar representing an optionally substituted aryl group, preferentially a phenyl optionally substituted with one or more alkyl, (O)₂S(O⁻)—, M⁺ or phenylamino groups;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)—; ix) R^{∘}—X′—C(X)—X″—; x) Ar—N═N— and xi) optionally substituted aryl(alkyl)amino; with M+, R^{∘}, X, X′, X″ and Ar as defined previously;
W represents a sigma bond σ, an oxygen or sulfur atom, or a divalent radical i) —NR— with R as defined previously, or ii) methylene —C(Ra)(Rb)— with Ra and Rb, which may be identical or different, representing a hydrogen atom or an aryl group, or alternatively Ra and Rb together form, with the carbon atom that bears them, a spiro cycloalkyl; preferentially, W represents a sulfur atom or Ra and Rb together form a cyclohexyl; it being understood that formulae (XI) and (XI′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (XI), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2; Food Yellow 3 or sunset yellow; and as examples of dyes of formula (XI′), mention may be made of: Acid Red 111, Acid Red 134 or Acid Yellow 38;

b) the pyrazolone anionic azo dyes of formulae (XII) and (XII′):

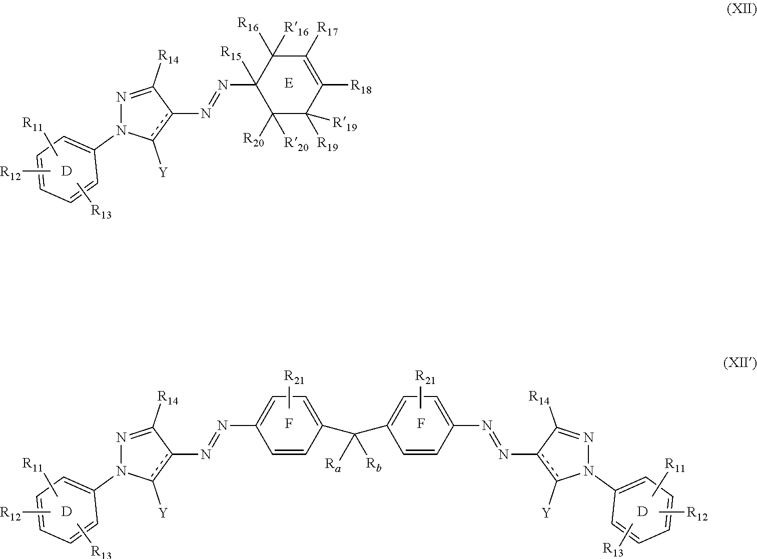

formulae (XII) and (XII′) wherein:
R₁₁, R₁₂ and R₁₃, which may be identical or different, represent a hydrogen or halogen atom, an alkyl group or —(O)₂S(O⁻), M⁺ with M⁺ as defined previously;
R₁₄ represents a hydrogen atom, an alkyl group or a group —C(O)O—, M⁺ with M⁺ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, or a group chosen from:
(O)₂S(O⁻)—, M⁺ with M⁺ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Ra and Rb, which may be identical or different, are as defined previously; preferentially, Ra represents a hydrogen atom and Rb represents an aryl group;
Y represents either a hydroxyl group or an oxo group;
[symbol] represents a single bond when Y is an oxo group; and represents a double bond when Y represents a hydroxyl group;
   it being understood that formulae (XII) and (XII′) comprise at least one sulfonate radical (O)₂S(O⁻)—, M⁺ or one carboxylate radical —C(O)O—, M⁺ on one of the rings D or E;
   preferentially sodium sulfonate.

As examples of dyes of formula (XII), mention may be made of: Acid Red 195, Acid Yellow 23, Acid Yellow 27, Acid Yellow 76, and as an example of a dye of formula (XII′), mention may be made of: Acid Yellow 17;

c) the anthraquinone dyes of formulae (XIII) and (XIII′):

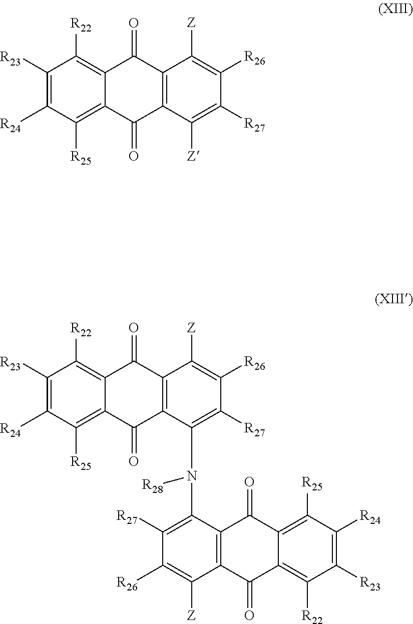

formulae (XIII) and (XIII′) wherein:
R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or arylthio, preferentially substituted with one or more groups chosen from alkyl and (O)₂S(O⁻)—, M⁺ with M⁺ as defined previously;
aryl(alkyl)amino optionally substituted with one or more groups chosen from alkyl and (O)₂S(O⁻)—, M⁺ with M⁺ as defined previously;
(di)(alkyl)amino;
(di)(hydroxyalkyl)amino;
(O)₂S(O⁻)—, M⁺ with M⁺ as defined previously;
Z′ represents a hydrogen atom or a group NR₂₈R²⁹ with R₂₈ and R₂₉, which may be identical or different, representing a hydrogen atom or a group chosen from:
alkyl;
polyhydroxyalkyl such as hydroxyethyl;
aryl optionally substituted with one or more groups, particularly i) alkyl such as methyl, n-dodecyl, n-butyl; ii) (O)₂S(O⁻)—, M⁺ with M⁺ as defined previously; iii) R^{∘}—C(X)—X′—, R^{∘}—X′—C(X)—, R^{∘}—X′—C(X)—X″— with R^{∘}, X, X′ and X″ as defined previously, preferentially R^{∘} represents an alkyl group;
cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
   it being understood that formulae (XIII) and (XIII′) comprise at least one sulfonate radical (O)₂S(O⁻)—, M⁺ or one carboxylate radical —C(O)O—, M⁺; preferentially sodium sulfonate.

As examples of dyes of formula (XIII), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT violet No. 2; and, as an example of a dye of formula (XIII′), mention may be made of: Acid Black 48;

d) the nitro dyes of formulae (XIV) and (XIV′):

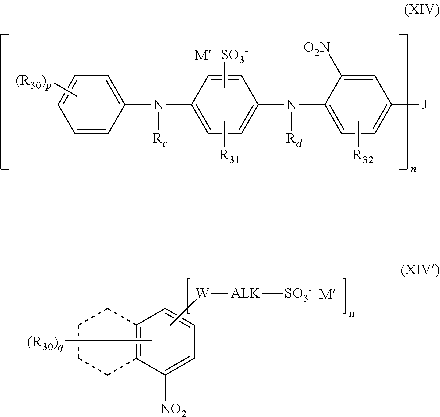

in which formulae (XIV) and (XIV′):
R³⁰, R₃₁ and R³², which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R^{∘}—C(X)—X′—, R^{∘}—X′—C(X)—, R^{∘}—X′—C(X)—X″— with R^{∘}, X, X′ and X″ as defined previously;
(O)₂S(O⁻)—, M⁺ with M⁺ as defined previously;
(O)CO⁻—, M⁺ with M⁺ as defined previously;
(di)(alkyl)amino;
(di)(hydroxyalkyl)amino;
heterocycloalkyl such as piperidino, piperazino or morpholino; in particular, R₃₀, R₃₁ and R₃₂ represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a —CH₂—CH₂— group;
n is 1 or 2;
p represents an integer inclusively between 1 and 5;
q represents an integer inclusively between 1 and 4;
u is 0 or 1;
when n is 1, J represents a nitro or nitroso group; particularly nitro;
when n is 2, J represents an oxygen or sulfur atom, or a divalent radical —S(O)ₘ— with m representing an integer 1 or 2; preferentially, J represents an —SO₂— radical;
M′ represents a hydrogen atom or a cationic counterion;

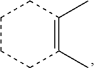

which may be present or absent, represents a benzo group optionally substituted with one or more groups R₃₀ as defined previously, it being understood that formulae (XIV) and (XIV′) comprise at least one sulfonate radical (O)₂S(O⁻)—, M⁺ or one carboxylate radical —C(O)O⁻, M⁺; preferentially sodium sulfonate.

As examples of dyes of formula (XIV), mention may be made of: Acid Brown 13 and Acid Orange 3; as examples of dyes of formula (XIV′), mention may be made of: Acid Yellow 1, the sodium salt of 2,4-dinitro-1-naphthol-7-sulfonic acid, 2-piperidino-5-nitrobenzenesulfonic acid, 2-(4′—N,N(2″-hydroxyethyl)amino-2′-nitro)anilineethanesulfonic acid, 4-p-hydroxyethylamino-3-nitrobenzenesulfonic acid; EXT D&C Yellow 7;

e) the triarylmethane dyes of formula (XV):

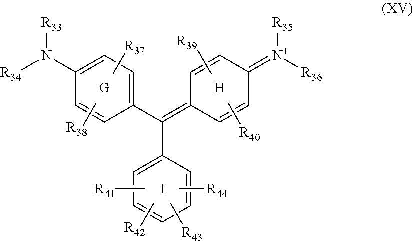

formula (XV) wherein:
R₃₃, R³⁴, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
R₃₇, R₃₈, R₃₉, R₄₀, R₄₁, R₄₂, R₄₃ and R⁴⁴, which may be identical or different, represent a hydrogen atom or a group chosen from:
alkyl;
alkoxy, alkylthio;
(di)(alkyl)amino;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)— and ix) R^{∘}—X′—C(X)—X″—; with M⁺, R^{∘}, X, X′ and X″ as defined previously;
   in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
   it being understood that at least one of the rings G, H, I or I′ comprises at least one sulfonate radical (O)₂S(O⁻)— or one carboxylate radical —C(O)O—; preferentially sulfonate. As examples of dyes of formula (XV), mention may be made of: Acid Blue 1, Acid Blue 3, Acid Blue 7, Acid Blue 9, Acid Violet 49, Acid Green 3, Acid Green 5 and Acid Green 50;
   f) the xanthene-based dyes of formula (XVI):

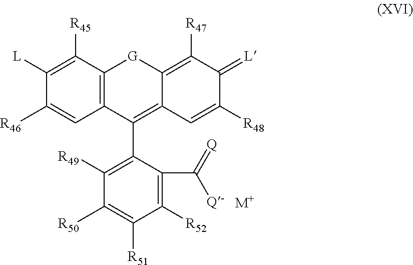

formula (XVI) wherein:
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined previously;
   particularly, R₅₃, R₅₄, R₅₅ and R₄₈ represent a hydrogen or halogen atom;
G represents an oxygen or sulfur atom or a group NRe with Re as defined previously; particularly G represents an oxygen atom;
L represents an alkoxide O⁻, M⁺; a thioalkoxide S⁻, M⁺ or a group NRf, with Rf representing a hydrogen atom or an alkyl group, and M⁺ as defined previously; M⁺is particularly sodium or potassium;
L′ represents an oxygen or sulfur atom or an ammonium group: N⁺ RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly, Q and Q′ represent an oxygen atom;
M⁺ is as defined previously.

As examples of dyes of formula (XVI), mention may be made of: Acid Yellow 73, Acid Red 51, Acid Red 52, Acid Red 87, Acid Red 92, Acid Red 95 and Acid Violet 9;

g) the indole-based dyes of formula (XVII):

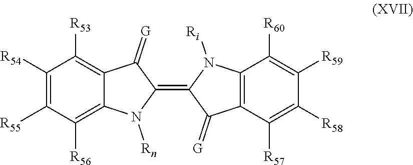

R₅₃, R₅₄, R₅₅, R₅₆, R₅₇, R₅₈, R₅₉ and R₆₀, which may be identical or different, represent a hydrogen atom or a group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
G represents an oxygen or sulfur atom or a group NRe with Re as defined previously; particularly G represents an oxygen atom;
Ri and Rh, which may be identical or different, represent a hydrogen atom or an alkyl group;
   it being understood that formula (XVII) comprises at least one sulfonate radical (O)₂S(O⁻)—, M⁺ or one carboxylate radical —C(O)O⁻, M⁺; preferentially sodium sulfonate.

As an example of a dye of formula (XVII), mention may be made of: Acid Blue 74.

h) the quinoline-based dyes of formula (XVIII):

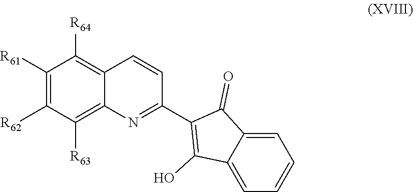

R₆₁ represents a hydrogen or halogen atom or an alkyl group;
R₆₂, R₆₃ and R₆₄, which may be identical or different, represent a hydrogen atom or a group (O)₂S(O⁻)—, M⁺ with M⁺ representing a hydrogen atom or a cationic counterion; or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
   it being understood that formula (XVIII) comprises at least one sulfonate radical (O)₂S(O⁻)—, M⁺, preferentially sodium sulfonate.

As examples of dyes of formula (XVIII), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The coloring agent(s) can be present in a total amount ranging from 0.001% to 15% by weight, preferably from 0.005% to 10% by weight, with respect to the total weight of the composition.

The pigments can be present in a total amount ranging from 0.05% to 15% by weight, preferably from 0.1% to 10% by weight, with respect to the total weight of the composition.

The direct dye(s) can be present in a total amount ranging from 0.001% to 10% by weight of the total weight of the composition, preferably from 0.005% to 5% by weight of the total weight of the composition.

### Metallic Compound:

The composition according to the invention may comprise at least one metallic compound.

For the purposes of the invention, the expression “metallic compound” is understood to mean a compound comprising at least one atom chosen from metals.

Preferably, the composition comprises at least one metallic compound, the metal(s) being chosen from transition metals, metals from the group of the lanthanide family, aluminum, boron, tin, bismuth, alkali and alkaline-earth metals such as magnesium, and mixtures thereof.

According to one preferred embodiment, the metallic compound(s) comprise(s) at least one metal chosen from titanium, tin, aluminum, zirconium, and mixtures thereof, better still titanium, aluminum and mixtures thereof.

The metallic compound(s) are different from the coloring agents as described below.

Preferably, the metallic compound(s) are chosen from organometallic compounds. The organometallic compound(s) is (are) preferably chosen from the metal alkoxides of formulae (XIXa), (XIXb), (XIXc) and (XIXd) below, and mixtures thereof:

M—(OR₁)ₙ (XIXa)

R-M(OR₁)ₙ₋₁ (XIXb)

(R₁O)ₙ₋₁-M-R″-M′—(OR₁)ₙ₋₁ (XIXc)

RR′-M—(OR₁)ₙ₋₂ (XIXd)

wherein:
M and M′, independently of one another, represent an atom chosen from transition metals, metals from the lanthanide group of the Periodic Table, aluminum, boron, tin, bismuth, alkali and alkaline-earth metals such as magnesium, preferably titanium or aluminum,
n represents the valency of the metal, and
R₁, which may be identical or different, represent a linear or branched, saturated or unsaturated hydrocarbon-based group containing from 1 to 30 carbon atoms, preferably from 1 to 6 carbon atoms, optionally interrupted with 1 to 20 heteroatoms chosen from O, N, S and/or P,
R and R′, independently of one another, represent a hydrogen atom or a linear, branched or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P,
R″ represents —O—, —NR₂—, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and/or P, with R₂ representing a linear, cyclic or branched, saturated or unsaturated hydrocarbon-based group containing from 1 to 30 carbon atoms, preferably from 2 to 20 carbon atoms.

More preferentially, the organometallic compound(s) is (are) chosen from metal alkoxides of formula (XIXa), wherein:
M represents an atom chosen from transition metals, metals from the lanthanide group of the Periodic Table, aluminum, boron, tin, bismuth, alkali and alkaline-earth metals such as magnesium, preferably titanium or aluminum;
n represents the valency of the atom;
R1, which may be identical or different, represent a linear or branched, saturated hydrocarbon-based group containing from 1 to 30 carbon atoms, preferably from 1 to 6 carbon atoms.

Even more preferentially, the organometallic compound(s) is (are) chosen from metal alkoxides of formula (XIXa), wherein:
M represents an atom chosen from titanium, tin, bismuth, aluminum and zirconium, preferably titanium or aluminum;
n represents the valency of the atom;
R₁ represents a methyl, ethyl, 2-ethylhexyl, propyl, isopropyl, n-butyl, isobutyl or t-butyl group.

According to one preferred embodiment, the organometallic compound(s) is (are) chosen from the tetraalkoxytitanium compounds of the following formula (XX):

Ti(OR₃)_{y}(OR₄)_{4-y} (XX)

wherein:
R³ and R₄ represent, independently of one another, a linear or branched alkyl radical containing from 1 to 30 carbon atoms, preferably from 1 to 6 carbon atoms, and
y is an integer ranging from 3 to 4.

As metallic compound that can be used in the present invention, mention may be made of organometallic compounds based on aluminum and notably aluminum di-s-butoxide ethylacetoacetate, organometallic compounds based on tin and notably tin bis(2-ethylhexanoate), tin bis(neodecanoate), di-n-butyltin bis(2,4-pentanedionate), di-n-butyltin diacetate, dimethyltin dineodecanoate, dimethylhydroxy(oelate)tin, dioctyllauryltin, dioctyldilauryltin, tin II oleate and mixtures thereof, organometallic compounds based on zirconium and notably zirconium ethoxide, zirconium propoxide, zirconium isopropoxide, zirconium butoxide, zirconium tert-butoxide, organometallic compounds based on titanium and notably titanium ethoxide, titanium propoxide, titanium isopropoxide, titanium butoxide, titanium tert-butoxide, titanium 2-ethylhexyloxide, and mixtures thereof.

Preferably, the metallic compound is titanium butoxide.

Titanium butoxide may for example be purchased from SHIN ETSU under the name D-25.

According to one embodiment, the metallic compound is a metal salt.

According to a preferred embodiment, the metal salt(s) are chosen from the salts of transition metals, the salts of alkali metals, the salts of alkaline-earth metals such as magnesium salts, aluminum salts, boron salts, tin salts, hydrates thereof and mixtures thereof, more preferentially titanium salts, aluminum salts, hydrates thereof and mixtures thereof.

For the purposes of the present invention, the term “metal salt” means a salt derived in particular from the action of at least one acid on a metal.

The metal salt(s) may be in the form of hydrates.

The metal salt(s) may be organic or inorganic.

The term “organic metal salt” is understood to mean a salt derived in particular from the action of an organic acid on a metal.

The term “inorganic metal salt” is understood to mean a salt derived in particular from the action of an inorganic acid on a metal.

The term “inorganic acid” means an acid which does not include any carbon atoms, apart from carbonic acid.

According to a particular embodiment of the invention, the inorganic metal salt(s) are chosen from halides such as chlorides, fluorides, iodides and bromides, carbonates, sulfates, phosphates, nitrates, perchlorates, hydrates thereof, and mixtures thereof. According to a particular embodiment of the invention, the inorganic metal salt(s) are chosen from aluminum sulfate, aluminum chloride, hydrated or non-hydrated, and mixtures thereof.

Preferentially, the metal salt(s) are organic, more preferentially chosen from citrates, lactates, glycolates, gluconates, acetates, propionates, fumarates, oxalates, glycinates, tartrates, benzoates, hydrates thereof and mixtures thereof.

According to a particularly preferred embodiment, the metal salt(s) are chosen from titanium citrate, aluminum acetate, aluminum lactate, hydrated or non-hydrated aluminum citrate, aluminum glycinate, aluminum benzoate and mixtures thereof.

The metallic compound(s) may be present in a total amount ranging from 0.01% to 10% by weight, preferably from 0.05% to 5% by weight, more preferentially from 0.1% to 3% by weight, relative to the total weight of the composition.

### Anhydrous Composition:

Preferably, the composition according to the present invention is an anhydrous composition.

The term “anhydrous composition” means a composition having a water content of less than 3% by weight, preferably less than 2% by weight and more preferably less than 1% by weight relative to the total weight of the composition.

More preferentially, the composition is free of water (zero content). The water is not added during the preparation of the composition, but may correspond to the residual water provided by the mixed ingredients.

According to another embodiment, the composition may comprise water. According to this embodiment, preferably, the composition comprises from 3% to 30% by weight of water relative to the total weight of the composition.

### Organic Solvents:

The composition according to the invention may comprise one or more organic solvents.

As organic solvent, mention may for example be made of alkanes containing from 8 to 16 carbon atoms, and notably branched C₈ to C₁₆ alkanes such as C₈ to C₁₆ isoalkanes of petroleum origin (also called isoparaffins), such as isododecane (also called 2,2,4,4,6-pentamethylheptane), isodecane or isohexadecane.

Mention may also be made of cyclic silicones, other than the silicones of formula (I) and the silicones of formula (II) mentioned above.

Preferably, the cyclic silicone is chosen from polydialkylsiloxanes, in particular polydimethylsiloxanes (PDMSs), and organomodified polysiloxanes comprising at least one functional group chosen from aryl groups and alkoxy groups.

Organopolysiloxanes are defined in greater detail in Walter Noll's “Chemistry and Technology of Silicones” (1968), Academic Press. They may be volatile or non-volatile.

Preferably, the cyclic silicones are volatile.

When they are volatile, the cyclic silicones can be more particularly chosen from those having a boiling point of between 60° C. and 260° C., and even more particularly from cyclic polydialkylsiloxanes containing from 3 to 7 and preferably 4 to 5 silicon atoms. These are, for example, octamethylcyclotetrasiloxane sold notably under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by Union Carbide, of formula:

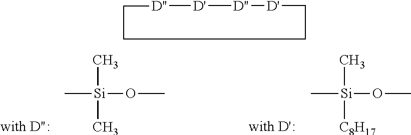

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane.

Advantageously, the cyclic silicones are chosen from cyclic polydialkylsiloxanes comprising from 3 to 7 and preferably from 4 to 5 silicon atoms, as mentioned above.

Preferably, the organic solvent(s) are chosen from C₈ to C₁₆ branched alkanes, cyclic silicones and mixtures thereof, more particularly from cyclic polydialkylsiloxanes comprising from 3 to 7 silicon atoms, better still from 4 to 5 silicon atoms.

The organic solvents may be present in a total amount ranging from 40% to 95% by weight relative to the total weight of the composition, preferably from 50% to 90% by weight, more preferentially from 70% to 90% by weight relative to the total weight of the composition according to the invention.

### Additives:

The composition may also contain any adjuvant or additive usually used.

Mention may be made, among the additives capable of being contained in the composition, of reducing agents, thickeners, softeners, antifoams, moisturizers, UV-screening agents, peptizers, solubilizers, fragrances, anionic, cationic, nonionic or amphoteric surfactants, proteins, vitamins, polymers other than the silicones previously described, preservatives, waxes and mixtures thereof.

The composition according to the invention may notably be in the form of a suspension, a dispersion, a gel, an emulsion, notably an oil-in-water (O/W) or water-in-oil (W/O) emulsion, or a multiple emulsion (W/0/W or polyol/0/W or O/W/O), in the form of a cream, a mousse, a stick, a dispersion of vesicles, notably of ionic or nonionic lipids, a two-phase or multi-phase lotion, an anhydrous liquid or an anhydrous gel.

According to one preferred embodiment, the composition according to the invention is an anhydrous liquid or an anhydrous gel.

Those skilled in the art may select the appropriate presentation form, and also the method for preparing it, on the basis of their general knowledge, taking into account firstly the nature of the constituents used, in particular their solubility in the support, and secondly the intended application of the composition.

According to a preferred embodiment, the composition according to the invention comprises at least one silicone of formula (I) as described previously, at least one silicone of formula (II) as described previously, and at least one pigment.

According to a more preferred embodiment, the composition according to the invention comprises at least one silicone of formula (Ia) as described previously, at least one silicone of formula (II) as described previously, and at least one pigment.

According to a more preferred embodiment, the composition according to the invention comprises at least one silicone of formula (Ia) as described previously, at least one silicone of formula (II) as described previously, at least one pigment and at least one alkoxysilane of formula (IIIa) as described previously.

According to a more preferred embodiment, the composition according to the invention comprises at least one silicone of formula (Ia) as described previously, at least one silicone of formula (II) as described previously, at least one pigment and APTES.

According to a more preferred embodiment, the composition according to the invention comprises at least one silicone of formula (Ia) as described previously, at least one silicone of formula (II) as described previously, at least one organometallic compound chosen from the tetraalkoxytitanium compounds of formula (XX) as described previously and at least one pigment.

According to a more preferred embodiment, the composition according to the invention comprises at least one silicone of formula (Ia) as described previously, at least one silicone of formula (II), as described previously, at least one organometallic compound chosen from the tetraalkoxytitanium compounds of formula (XX) as described previously, at least one pigment and at least one alkoxysilane of formula (IIIa) as described previously.

According to a more preferred embodiment, the composition according to the invention comprises at least one silicone of formula (Ia) as described previously, at least one silicone of formula (II) as described previously, titanium tetrabutoxide, at least one pigment and APTES.

### Use

The present invention also relates to the use of the composition as described previously for treating keratin fibers such as the hair, in particular for dyeing keratin fibers such as the hair.

### Method for Treating Keratin Fibers

The present invention also relates to a method for treating, in particular for dyeing, keratin fibers, such as the hair, wherein the composition according to the invention as described previously is applied to said fibers.

### Treatment Step:

Preferably, the method for treating keratin fibers such as the hair involves a step of treating the keratin fibers which consists in applying to said fibers a composition (G) comprising at least one alkoxysilane chosen from the compounds of formula (III), oligomers thereof and/or mixtures thereof as described previously.

According to a particular embodiment, the composition according to the invention and composition (G) are applied simultaneously to the keratin fibers.

According to another particular embodiment, composition (G) is applied to the keratin fibers before applying the composition according to the invention to the keratin fibers.

According to another particular embodiment, composition (G) is applied to the keratin fibers after applying the composition according to the invention to the keratin fibers.

Preferably, composition (G) is applied to the keratin fibers before applying the composition according to the invention to the keratin fibers.

In particular, the method for treating keratin fibers such as the hair comprises:
i) the application to said keratin fibers of a composition (G) comprising at least one alkoxysilane chosen from the compounds of formula (III), oligomers thereof and/or mixtures thereof as described previously,
ii) optionally a leave-on time of from 30 seconds to 20 minutes, notably from 30 seconds to 10 minutes, preferably from 30 seconds to 5 minutes, of composition (G) on said fibers,
iii) optionally a step of washing, rinsing, draining or drying said fibers,
iv) the application of the composition according to the invention as described previously,
v) optionally a leave-on time of from 30 seconds to 20 minutes, notably from 30 seconds to 10 minutes, preferably from 30 seconds to 5 minutes, of the composition according to the invention on said fibers, and
vi) optionally a step of drying and/or heating the keratin fibers for example at a temperature above or equal to 30° C.

Preferably, a washing, rinsing, drying and/or draining step is performed after applying composition (G) to the keratin fibers and before applying the composition according to the invention to the keratin fibers.

The term “rinsing step” means a step of rinsing with water.

More preferentially, a drying step is performed after applying composition (G) to the keratin fibers and before applying the composition according to the invention to the keratin fibers.

The composition (G) comprises at least one alkoxysilane chosen from the compounds of formula (III), oligomers thereof and/or mixtures thereof as described previously.

The alkoxysilane(s) of formula (III), oligomers thereof and/or mixtures thereof may be present in a total amount ranging from 0.01% to 10% by weight, preferably from 0.05% to 5% by weight and better still from 0.07% to 3% by weight, relative to the total weight of the composition (G).

The composition (G) can comprise water. Preferably, the composition (G) comprises a total amount of water ranging from 30% to 99% by weight, relative to the total weight of the composition (G).

### Protocol:

Preferably, the composition according to the invention is a composition for dyeing keratin fibers, such as the hair.

The composition according to the invention and/or the composition (G) can be used on wet or dry keratin fibers, and also on any type of fair or dark, natural or dyed, permanent-waved, bleached or relaxed fibers.

According to a particular embodiment of the method of the invention, the fibers are washed before applying the composition according to the invention and/or the composition (G).

The application of the dye composition according to the invention and/or of the composition (G) to the keratin fibers may be performed by any conventional means, in particular using a comb, a fine brush, a coarse brush or with the fingers.

The dyeing process, i.e. application of the composition according to the invention and/or of the composition (G) to the keratin fibers, is generally carried out at ambient temperature (between 15° C. and 25° C.).

Preferably, after application of the composition according to the invention and before an optional step of applying heat to the keratin fibers, the keratin fibers are subjected to a drying step, for example, using a hairdryer, for a time of between 1 minute and 5 minutes. Preferably, after application of the composition according to the invention to the keratin fibers, there is a waiting time of at least 30 seconds, preferably of at least 1 minute, before the step of drying the keratin fibers.

According to a particular embodiment, there is no leave-on time between applying the composition according to the invention to the keratin fibers and the step of drying the keratin fibers.

The drying step may be performed using absorbent paper, a hairdryer or a styling hood or by drying naturally.

After application of the composition according to the invention and optionally a step of drying the keratin fibers, the method according to the invention may comprise a step of applying heat to the keratin fibers, using a heating tool.

The heat application step of the method of the invention may be performed using a hood, a hairdryer, a straightening iron, a curling iron, a Climazon, etc.

Preferably, the heat application step of the method of the invention is performed using a hairdryer and/or a straightening iron, more preferentially using a straightening iron.

During the step of applying heat to the keratin fibers, a mechanical action may be exerted on the locks, such as combing, brushing or running the fingers through.

When the step of applying heat to the keratin fibers is performed using a hood or a hairdryer, the temperature is preferably between 30° C. and 110° C., preferentially between 50° C. and 90° C.

When the step of applying heat to the keratin fibers is performed using a straightening iron, the temperature is preferably between 110° C. and 220° C., preferably between 140° C. and 200° C.

In a particular variant, the method of the invention involves a step (b1) of applying heat using a hood, a hairdryer or a Climazon, preferably a hairdryer, and a step (b2) of applying heat using a straightening or curling iron, preferably a straightening iron.

Step (b1) may be performed before step (b2).

During step (b1), also referred to as the drying step, the fibers may be dried, for example at a temperature above or equal to 30° C. According to a particular embodiment, this temperature is above 40° C. According to a particular embodiment, this temperature is above 45° C. and below 110° C.

Preferably, if the fibers are dried, they are dried, in addition to a supply of heat, with a flow of air. This flow of air during drying makes it possible to improve the strand separation of the coating.

During drying, a mechanical action may be exerted on the locks, such as combing, brushing or running the fingers through.

During step (b2), the passage of the straightening or curling iron, preferably the straightening iron, can be carried out at a temperature ranging from 110° C. to 220° C., preferably between 140° C. and 200° C.

Preferably, after application of the composition according to the invention to the keratin fibers, there is a waiting time of at least 30 minutes before the first shampooing operation, preferably at least 1 h before the first shampooing operation, more preferentially at least 10 h before the first shampooing operation, better still at least 24 h before the first shampooing operation.

According to a preferred embodiment, the invention is a method for treating keratin fibers such as the hair, comprising:
i) the application to said fibers of a composition (G) comprising at least one alkoxysilane chosen from the compounds of formula (III), oligomers thereof and/or mixtures thereof as described previously, then
ii) optionally a leave-on time of from 30 seconds to 20 minutes, notably from 30 seconds to 10 minutes, preferably from 30 seconds to 5 minutes, of composition (G) on said fibers, then
iii) optionally a step of washing, rinsing, draining or drying said fibers, then
iv) the application to said fibers of the composition according to the invention comprising:
a) at least one silicone of formula (I) as described previously,
b) at least one silicone of formula (II) as described previously, and
c) at least one coloring agent chosen from pigments, direct dyes and mixtures thereof,
d) optionally at least one metallic compound, then
v) optionally a leave-on time of from 30 seconds to 20 minutes, notably from 30 seconds to 10 minutes, preferably from 30 seconds to 5 minutes, of the composition according to the invention on said fibers, then
vi) optionally a step of drying and/or heating the keratin fibers for example at a temperature above or equal to 30° C.

### Method for Treating Keratin Fibers

The present invention also relates to a method for treating keratin fibers such as the hair, comprising:
(i) a step of applying to the keratin fibers a composition (A) comprising at least one metallic compound as described previously; and
(ii) a step of applying to the keratin fibers a composition (B) comprising:
at least one silicone of formula (I) as described previously, and
at least one silicone of formula (II) as described previously,
   the composition (A) and/or the composition (B) comprising at least one coloring agent chosen from pigments, direct dyes and mixtures thereof as described previously; it being understood that steps i) and ii) are performed successively i), then ii) or else ii), then i).

Composition (A) according to the invention comprises at least one metallic compound as described previously.

The metallic compound(s) may be present in a total amount ranging from 0.01% to 10% by weight, preferably from 0.05% to 5% by weight, more preferentially from 0.1% to 3% by weight, relative to the total weight of composition (A).

Composition (A) according to the invention may comprise one or more organic solvents as described previously.

The organic solvents may be present in a total amount ranging from 40% to 99.8% by weight relative to the total weight of composition (A), preferably from 50% to 99.7% by weight relative to the total weight of composition (A).

Composition (B) according to the invention comprises at least one silicone of formula (I) as described previously,

The silicone(s) of formula (I) may be present in a total amount ranging from 0.1% to 30% by weight, preferably from 1% to 20% by weight and more preferentially from 1% to 15% by weight relative to the total weight of composition (B).

Composition (B) according to the invention comprises at least one silicone of formula (II) as described previously.

The silicone(s) of formula (II) may be present in a total amount ranging from 0.1% to 30% by weight, preferably from 1% to 25% by weight and more preferentially from 1% to 20% by weight, better still from 3% to 15% by weight relative to the total weight of composition (B).

Composition (B) according to the invention may comprise one or more organic solvents as described previously.

The organic solvents may be present in a total amount ranging from 40% to 95% by weight relative to the total weight of composition (B), preferably from 50% to 90% by weight, more preferentially from 70% to 90% by weight relative to the total weight of composition (B).

Composition (B) may comprise a silicone of formula (I)/silicone of formula (II) weight ratio ranging from 95:5 to 5:95.

Preferably, the silicone of formula (I)/silicone of formula (II) weight ratio varies from 90:10 to 10:90, preferentially from 80:20 to 20:80 and more preferentially from 70:30 to 30:70.

According to one particular embodiment, the silicone of formula (Ia)/silicone of formula (II) weight ratio varies from 90:10 to 10:90, preferentially from 80:20 to 20:80 and more preferentially from 70:30 to 30:70.

Preferably, composition (A) and/or composition (B) comprises one or more pigments. According to a preferred embodiment, composition (B) comprises at least one coloring agent chosen from pigments, direct dyes and mixtures thereof, more preferentially one or more pigments.

According to a preferred embodiment, composition (A) does not comprise a coloring agent chosen from pigments, direct dyes and mixtures thereof.

The coloring agent(s) may be present in a total amount ranging from 0.001% to 15%, preferably from 0.005% to 10% by weight relative to the total weight of composition (A) or composition (B).

The pigments may be present in a total amount ranging from 0.05% to 15% by weight, preferably from 0.1% to 10% by weight relative to the total weight of composition (A) or composition (B).

The direct dye(s) may be present in a total amount ranging from 0.001% to 10% by weight of the total weight of the composition, preferably from 0.005% to 5% by weight of the total weight of composition (A) or of composition (B).

Composition (A) and/or composition (B) may comprise at least one alkoxysilane chosen from the compounds of formula (III), oligomers thereof and/or mixtures thereof as described previously.

The alkoxysilane(s) of formula (III), oligomers thereof and/or mixtures thereof may be present in a total amount ranging from 0.01% to 10% by weight, preferably from 0.05% to 5% by weight and better still from 0.07% to 3% by weight, relative to the total weight of composition (A) or of composition (B).

Preferably, composition (A) according to the present invention is an anhydrous composition.

Preferably, composition (B) according to the present invention is an anhydrous composition.

The term “anhydrous composition” means a composition having a water content of less than 3% by weight, preferably less than 2% by weight and more preferably less than 1% by weight relative to the total weight of the composition.

More preferentially, composition (A) and/or composition (B) is (are) free of water (zero content). The water is not added during the preparation of the composition, but may correspond to the residual water provided by the mixed ingredients.

According to another embodiment, composition (A) and/or composition (B) may comprise water. According to this embodiment, preferably, the composition comprises from 3% to 30% by weight of water relative to the total weight of the composition. Composition (A) and/or composition (B) may also contain any adjuvant or additive customarily used as described previously.

Composition (A) and/or composition (B) may notably be in the form of a suspension, a dispersion, a gel, an emulsion, notably an oil-in-water (O/W) or water-in-oil (W/O) emulsion, or a multiple emulsion (W/O/W or polyol/O/W or O/W/O), in the form of a cream, a mousse, a stick, a dispersion of vesicles, notably of ionic or nonionic lipids, a two-phase or multi-phase lotion, an anhydrous liquid or an anhydrous gel.

According to a preferred embodiment, composition (A) according to the invention is an anhydrous liquid or an anhydrous gel.

According to a preferred embodiment, composition (B) according to the invention is an anhydrous liquid or an anhydrous gel.

According to a preferred embodiment, composition (B) according to the invention comprises at least one silicone of formula (I) as described previously, at least one silicone of formula (II) as described previously, and at least one pigment.

According to a more preferred embodiment, composition (B) according to the invention comprises at least one silicone of formula (Ia) as described previously, at least one silicone of formula (II) as described previously, and at least one pigment.

According to a more preferred embodiment, composition (B) according to the invention comprises at least one silicone of formula (Ia) as described previously, at least one silicone of formula (II) as described previously, at least one pigment and at least one alkoxysilane of formula (IIIa) as described previously.

According to a more preferred embodiment, composition (B) according to the invention comprises at least one silicone of formula (Ia) as described previously, at least one silicone of formula (II) as described previously, at least one pigment and APTES.

Composition (A) and composition (B) are applied successively to the keratin fibers such as the hair.

According to a preferred embodiment, steps i) and ii) are carried out successively i), then ii).

Preferably, the composition (A) is applied to the keratin fibers such as the hair, followed by the application of composition (B) to the keratin fibers.

Composition (A) and/or composition (B) can be used on wet or dry keratin fibers, and also on any type of fair or dark, natural or dyed, permanent-waved, bleached or relaxed fibers.

According to a particular embodiment of the method of the invention, the fibers are washed before applying composition (A) and/or composition (B).

The application of composition (A) and/or of composition (B) to the keratin fibers may be carried out by any conventional means, in particular using a comb, a fine brush, a coarse brush or with the fingers.

The dyeing method, i.e. application of composition (A) and/or of composition (B) to the keratin fibers, is generally carried out at ambient temperature (between 15° C. and 25° C.).

Preferably, after application of composition (A) and/or of composition (B) and before an optional step of applying heat to the keratin fibers, the keratin fibers are subjected to a drying step, for example, using a hairdryer, for a time of between 1 minute and 5 minutes.

Preferably, after application of composition (B) according to the invention to the keratin fibers, there is a waiting time of at least 30 seconds, preferably of at least 1 minute, before the step of drying the keratin fibers.

According to a particular embodiment, there is no leave-on time between applying composition (B) to the keratin fibers and the step of drying the keratin fibers.

The drying step may be performed using absorbent paper, a hairdryer or a styling hood or by drying naturally.

After application of composition (A) and composition (B) according to the invention and optionally a step of drying the keratin fibers, the method according to the invention may comprise a step of applying heat to the keratin fibers, using a heating tool.

Preferably, the method according to the invention does not comprise a step of applying heat to the keratin fibers using a heating tool in addition to the optional drying step. The heat application step of the method of the invention may be performed using a hood, a hairdryer, a straightening iron, a curling iron, a Climazon, etc.

Preferably, the heat application step of the method of the invention is performed using a hairdryer and/or a straightening iron, more preferentially using a straightening iron. During the step of applying heat to the keratin fibers, a mechanical action may be exerted on the locks, such as combing, brushing or running the fingers through. When the step of applying heat to the keratin fibers is performed using a hood or a hairdryer, the temperature is preferably between 30° C. and 110° C., preferentially between 50° C. and 90° C.

When the step of applying heat to the keratin fibers is performed using a straightening iron, the temperature is preferably between 110° C. and 220° C., preferably between 140° C. and 200° C.

In a particular variant, the method of the invention involves a step (b1) of applying heat using a hood, a hairdryer or a Climazon, preferably a hairdryer, and a step (b2) of applying heat using a straightening or curling iron, preferably a straightening iron.

Step (b1) may be performed before step (b2).

During step (b1), also referred to as the drying step, the fibers may be dried, for example at a temperature above or equal to 30° C. According to a particular embodiment, this temperature is above 40° C. According to a particular embodiment, this temperature is above 45° C. and below 110° C.

Preferably, if the fibers are dried, they are dried, in addition to a supply of heat, with a flow of air. This flow of air during drying makes it possible to improve the strand separation of the coating.

During drying, a mechanical action may be exerted on the locks, such as combing, brushing or running the fingers through.

During step (b2), the passage of the straightening or curling iron, preferably the straightening iron, can be carried out at a temperature ranging from 110° C. to 220° C., preferably between 140° C. and 200° C.

Preferably, after application of composition (A) and composition (B) to the keratin fibers, there is a waiting time of at least 30 minutes before the first shampooing operation, preferably at least 1 h before the first shampooing operation, more preferentially at least 10 h before the first shampooing operation, better still at least 24 h before the first shampooing operation.

According to a preferred embodiment, the method for treating human keratin fibers, such as the hair, comprises the following successive steps:
i) the application to said fibers of a composition (A) comprising at least one metallic compound as described previously,
ii) optionally a step of washing, rinsing, draining or drying said fibers,
iii) the application of composition (B) comprising:
   at least one silicone of formula (I) as described previously, and
   at least one silicone of formula (II) as described previously, and
   at least one coloring agent chosen from pigments, direct dyes and mixtures thereof as described previously; and
iv) optionally a step of drying and/or heating the keratin fibers for example at a temperature above or equal to 30° C.

According to a preferred embodiment, the method for treating human keratin fibers, such as the hair, comprises the following successive steps:
i) the application to said fibers of a composition (A) comprising at least one metallic compound as described previously,
ii) optionally a leave-on time of from 30 seconds to 20 minutes, notably from 30 seconds to 10 minutes, preferably from 30 seconds to 5 minutes, of composition (A) on said fibers,
iii) optionally a step of washing, rinsing, draining or drying said fibers,
iv) the application of composition (B) comprising:
   at least one silicone of formula (I) as described previously, and
   at least one silicone of formula (II) as described previously, and
   at least one coloring agent chosen from pigments, direct dyes and mixtures thereof as described previously;
v) optionally a leave-on time of from 30 seconds to 20 minutes, notably from 30 seconds to 10 minutes, preferably from 30 seconds to 5 minutes, of composition (B) on said fibers, and
vi) optionally a step of drying and/or heating the keratin fibers for example at a temperature above or equal to 30° C.

Preferably, a washing, rinsing, drying and/or draining step is performed after applying composition (A) to the keratin fibers and before applying composition (B) to the keratin fibers.

The term “rinsing step” means a step of rinsing with water.

More preferentially, a drying step is performed after applying composition (A) to the keratin fibers and before applying composition (B) to the keratin fibers.

### Makeup-Removing Composition:

The process according to the invention may comprise a step of applying a makeup-removing composition to dyed keratin fibers such as the hair.

Preferably, the method according to the invention comprises a step of applying a makeup-removing composition to the keratin fibers after the application of the composition according to the invention and/or the application of composition (G) followed by the application of the composition according to the invention to the keratin fibers.

Preferably, the method according to the invention comprises a step of applying a makeup-removing composition to the keratin fibers after the application of composition (A) and the application of composition (B) to the keratin fibers.

The makeup-removing composition may correspond to a makeup-removing composition conventionally used in cosmetics.

Preferably the makeup-removing composition comprises at least one compound chosen from surfactants, solvents, fatty substances, acids, bases and mixtures thereof.

According to a preferred embodiment, the makeup-removing composition comprises at least one hydrocarbon-based oil.

Preferably, the hydrocarbon-based oil(s) are chosen from synthetic esters of formula R1COOR2 wherein R1 represents a fatty acid residue including from 8 to 29 carbon atoms, and R2 represents a branched or unbranched hydrocarbon-based chain containing from 3 to 30 carbon atoms, and mixtures thereof, more preferentially from isopropyl myristate, isononyl isononanoate and/or mixtures thereof.

The application of the dye composition to the keratin fibers such as the hair is carried out before the application of a makeup-removing composition.

The makeup-removing composition may be applied to keratin fibers dyed by the composition according to the invention as defined previously and/or the application of composition (G) followed by the application of the composition according to the invention as defined previously.

The step of applying heat to the keratin fibers may be performed after the application of the dye composition and before the application of the makeup-removing composition to the keratin fibers.

The application of the makeup-removing composition may be performed on dry dyed keratin fibers or wet dyed keratin fibers and also on all types of fibers.

The makeup-removing process is generally performed at ambient temperature (between 15° C. and 25° C.).

The application of the makeup-removing composition may be performed immediately after the application of the dye composition (i.e. a few minutes to a few hours after the application of the dye composition), or in the days or weeks following the application of the dye composition.

The makeup-removing composition may be applied with the aid of any suitable support, which is notably capable of absorbing it, for example a fibrous makeup-removing disk, for example a woven or nonwoven fabric, cotton wool, a flocked film, a sponge, a wipe, or a twisted or injection-molded mascara application brush.

The makeup-removing composition may be contained in a container and taken up for each makeup-removing operation. As a variant, the makeup-removing composition impregnates the support used for the makeup-removing operation, the support possibly being packaged, in this case, for example in leaktight packaging.

After the use of the makeup-removing composition, the keratin fibers may not be rinsed. As a variant, they may be rinsed. The rinsing may be performed, for example, with running water, without addition of a soap.

### Multi-Compartment Device (Kit)

The present invention also relates to a device for treating keratin fibers such as the hair, comprising one or more compartments containing:
in a first compartment, a composition according to the invention comprising:
   a) at least one silicone of formula (I) as described previously,
   b) at least one silicone of formula (II) as described previously, and
   c) at least one coloring agent chosen from pigments, direct dyes and mixtures thereof,
   d) optionally at least one metallic compound, and
optionally, in a second compartment, a composition (G) comprising at least one alkoxysilane chosen from the compounds of formula (III), oligomers thereof and/or mixtures thereof as described previously.

According to a preferred variant of the invention, the treating device according to the invention is a device for treating keratin fibers such as the hair, comprising one or more compartments containing:
in a first compartment, a composition according to the invention as described previously;
optionally, in a second compartment, a composition (G) as described previously; and
optionally, in a third compartment, a makeup-removing composition as described previously.

The present invention also relates to a device for treating keratin fibers such as the hair, comprising one or more compartments containing:
in a first compartment, a composition (A) comprising at least one metallic compound as described previously;
in a second compartment, a composition (B) comprising:
   at least one silicone of formula (I) as described previously, and
at least one silicone of formula (II) as described previously,
   the composition (A) and/or the composition (B) comprising at least one coloring agent chosen from pigments, direct dyes and mixtures thereof as described previously.

According to a preferred variant of the invention, the treating device according to the invention is a device for treating keratin fibers such as the hair, comprising one or more compartments containing:
in a first compartment, the composition (A) as described previously;
in a second compartment, a composition (B) as described previously; and
optionally, in a third compartment, a makeup-removing composition as described previously.

The present invention will now be described more specifically by means of examples, which are in no way limiting of the scope of the invention. However, the examples make it possible to support specific features, variants and preferred embodiments of the invention.

### EXAMPLES

### Example 1

The following composition is prepared (in g/100 g, AM: Active Material):

TABLE 1

| Composition | A |
|---|---|
| Dimethiconol⁽¹⁾ | 3.3 |
| Triethoxysilylethyl | 6.7 |
| Polydimethylsiloxyethyl dimethicone ⁽²⁾ | |
| 3-aminopropyltriethoxysilane ⁽³⁾ | 1.0 |
| Pigment (iron oxide sold by the company | 5.7 |
| Sun Chemical under the name SunPuro | |
| Red Iron Oxide ®) | |
| Cyclopentasiloxane | qs 100 |

⁽¹⁾sold under the trade name KF-9701 by Shin-Etsu;
⁽²⁾ sold under the trade name KF-9908 by Shin-Etsu;
⁽³⁾ sold under the trade name KBE-903 by Shin-Etsu.

### Protocol:

The composition A is applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

The locks of hair are left at 25° C. and 80% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described below.

### Shampoo Wash Protocol:

The locks of hair are washed with a standard shampoo (Garnier Ultra Doux).

The locks of hair are then rinsed, combed and dried with a hairdryer.

The next shampoo wash is performed on the locks obtained after the application of the hairdryer.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described above. The lower the ΔE value, the more persistent the color with respect to shampoo washing.

The ΔE value is calculated according to the following equation:

ΔE=\sqrt{(L*−L₀*)²+(a*−a₀*)²+(b*−b₀*)²} [Math.1]

In this equation, L*a*b* represent the values measured after dyeing the hair and after having undergone shampoo washes, and L₀*a₀*b₀* represent the values measured after dyeing the hair but before shampoo washing.

TABLE 2

| | Number of | | | | |
|---|---|---|---|---|---|
| | shampoo | | | | |
| Composition | washes | L* | a* | b* | ΔE |
| Composition A | 0 | 35.9 | 34.1 | 30.8 | — |
| (invention) | 3 | 62.9 | 7.9 | 23.2 | 38.4 |

The colored coating of the keratin fibers obtained with composition A according to the invention shows good persistence with respect to shampoo washes. Specifically, the locks of hair dyed with composition A according to the invention and washed with three shampoo washes show good persistence of the color.

### Example 2

The following composition is prepared (in g/100 g, AM: Active Material):

TABLE 3

| Composition | G |
|---|---|
| 3-aminopropyltriethoxysilane ⁽¹⁾ | 1.0 |
| Water | qs 100 |

⁽¹⁾ sold under the trade name KBE-903 by Shin-Etsu.

### Protocol:

The composition G is applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

Composition A as defined in example 1 is then applied to the locks of hair treated with composition G, in a proportion of 1 g of composition per gram of lock.

The locks of hair are left for 1 minute at ambient temperature.

The locks of hair are then combed and dried with a hairdryer for 3 minutes.

The locks of hair are left at 25° C. and 80% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described in example 1.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described in example 1. The lower the ΔE value, the more persistent the color with respect to shampoo washing.

The ΔE value is calculated according to the equation described in example 1.

TABLE 4

| | Number of | | | | |
|---|---|---|---|---|---|
| | shampoo | | | | |
| Composition | washes | L* | a* | b* | ΔE |
| Composition | 0 | 36.0 | 32.0 | 28.9 | — |
| G + A | 3 | 58.3 | 17.1 | 26.3 | 26.9 |
| (invention) | | | | | |

The colored coating of the keratin fibers obtained with composition G then composition A according to the invention shows good persistence with respect to shampoo washes. Specifically, the locks of hair dyed with composition G then the composition A according to the invention and washed with three shampoo washes show good persistence of the color.

### Example 3

The following compositions are prepared (in g/100 g, AM: Active Material):

TABLE 5

| Compositions | B | C |
|---|---|---|
| Dimethiconol⁽¹⁾ | 3.3 | 3.3 |
| Triethoxysilylethyl | 6.7 | 6.7 |
| Polydimethylsiloxyethyl | | |
| dimethicone ⁽²⁾ | | |
| Titanium butoxide ⁽³⁾ | 0.3 | — |
| Pigment (iron oxide sold by | 5.7 | 5.7 |
| the company Sun Chemical | | |
| under the name SunPuro Red | | |
| Iron Oxide ®) | | |
| Cyclopentasiloxane | qs 100 | qs 100 |

⁽¹⁾sold under the trade name KF-9701 by Shin-Etsu;
⁽²⁾ sold under the trade name KF-9908 by Shin-Etsu;
⁽³⁾ sold under the trade name D-25 by Shin-Etsu.

### Protocol:

Compositions B and C are applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

The locks of hair are left at 25° C. and 40% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described in example 1.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described above. The lower the ΔE value, the more persistent the color with respect to shampoo washing.

The ΔE value is calculated according to the equation described in example 1.

TABLE 6

| | Number of | | | | |
|---|---|---|---|---|---|
| | shampoo | | | | |
| Compositions | washes | L* | a* | b* | ΔE |
| Composition | 0 | 38.7 | 29.4 | 24.8 | — |
| B | 3 | 59.5 | 9.2 | 20.3 | 29.3 |
| Composition | 0 | 36.0 | 33.2 | 29.2 | — |
| C | 3 | 62.9 | 7.5 | 22.4 | 37.8 |

Thus, the colored coating of the keratin fibers obtained with composition C according to the invention shows good persistence with respect to shampoo washes. Specifically, the locks of hair dyed with composition C according to the invention and washed with three shampoo washes show good persistence of the color.

The colored coating of the keratin fibers obtained with composition B according to the invention, comprising at least one metallic compound, shows good persistence with respect to shampoo washes. Specifically, the locks of hair dyed with composition B according to the invention and washed with three shampoo washes show good persistence of the color.

### Example 4

The following composition is prepared (in g/100 g, AM: Active Material):

TABLE 7

| Compositions | D |
|---|---|
| Dimethiconol⁽¹⁾ | 3.3 |
| Triethoxysilylethyl Polydimethylsiloxyethyl dimethicone ⁽²⁾ | 6.7 |
| Titanium butoxide ⁽³⁾ | 0.3 |
| 3-aminopropyltriethoxysilane ⁽⁴⁾ | 1.0 |
| Pigment (iron oxide sold by the company Sun Chemical | 5.7 |
| under the name SunPuro Red Iron Oxide ®) | |
| Cyclopentasiloxane | qs 100 |

⁽¹⁾sold under the trade name KF-9701 by Shin-Etsu;
⁽²⁾ sold under the trade name KF-9908 by Shin-Etsu;
⁽³⁾ sold under the trade name D-25 by Shin-Etsu;
⁽⁴⁾ sold under the trade name KBE-903 by Shin-Etsu.

### Protocol:

Composition D is applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

The locks of hair are left at 25° C. and 40% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described in example 1.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described in example 1. The lower the ΔE value, the more persistent the color with respect to shampoo washing.

The ΔE value is calculated according to the equation described in example 1.

TABLE 8

| | Number of | | | | |
|---|---|---|---|---|---|
| | shampoo | | | | |
| Compositions | washes | L* | a* | b* | ΔE |
| Composition | 0 | 39.3 | 30.2 | 26.0 | — |
| D | 3 | 51.9 | 19.2 | 23.7 | 16.9 |

Thus, the colored coating of the keratin fibers obtained with composition D according to the invention shows good persistence with respect to shampoo washes. Specifically, the locks of hair dyed with composition D according to the invention and washed with three shampoo washes show good persistence of the color.

### Example 5

The composition G as described in example 2 is applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

Composition B as defined in example 3 is then applied to the locks of hair treated with composition G, in a proportion of 1 g of composition per gram of lock.

The locks of hair are left for 1 minute at ambient temperature.

The locks of hair are then combed and dried with a hairdryer for 3 minutes.

The locks of hair are left at 25° C. and 40% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described in example 1.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described in example 1. The lower the ΔE value, the more persistent the color with respect to shampoo washing.

The ΔE value is calculated according to the equation described in example 1.

TABLE 9

| | Number of | | | | |
|---|---|---|---|---|---|
| | shampoo | | | | |
| Composition | washes | L* | a* | b* | ΔE |
| Composition | 0 | 38.1 | 31.3 | 26.0 | — |
| G + B | 3 | 51.3 | 19.2 | 23.0 | 18.2 |

The colored coating of the keratin fibers obtained with composition G then composition B according to the invention shows good persistence with respect to shampoo washes. Specifically, the locks of hair dyed with composition G then the composition B according to the invention and washed with three shampoo washes show good persistence of the color.

### Example 6

The following compositions are prepared (in g/100 g, AM: Active Material):

TABLE 10

| Compositions | H |
|---|---|
| Titanium butoxide ⁽¹⁾ | 0.3 |
| Cyclopentasiloxane | qs 100 |

TABLE 11

| Compositions | I | J |
|---|---|---|
| Dimethiconol⁽²⁾ | 3.3 | 3.3 |
| Triethoxysilylethyl | 6.7 | 6.7 |
| Polydimethylsiloxyethyl dimethicone ⁽³⁾ | | |
| 3-aminopropyltriethoxysilane ⁽⁴⁾ | 1.0 | — |
| Pigment (iron oxide sold by the company | 5.7 | 5.7 |
| Sun Chemical under the name SunPuro | | |
| Red Iron Oxide ®) | | |
| Cyclopentasiloxane | qs 100 | qs 100 |

(1) sold under trade name D-25 by Shin-Etsu;
(2) sold under the trade name KF-9701 by Shin-Etsu;
(3) sold under the trade name KF-9908 by Shin-Etsu;
(4) sold under the trade name KBE-903 by Shin-Etsu;

### Protocol:

The composition H is applied to locks of dry natural hair having 90% white hairs, in a proportion of 1 g of composition per gram of lock.

The locks of hair are left for 1 minute at ambient temperature.

The locks of hair are then combed and dried with a hairdryer for 3 minutes.

Compositions I and J are then applied to the locks of hair treated with composition H in a proportion of 1 g of composition per gram of lock.

The locks of hair are left for 1 minute at ambient temperature.

The locks of hair are then combed and dried with a hairdryer for 3 minutes.

The locks of hair are left at 25° C. and 40% relative humidity for 24 h.

The locks of hair thus dyed are then subjected to a test of several repeated shampoo washes so as to evaluate the fastness (persistence) of the coloring obtained with respect to shampoo washes, according to the shampoo wash protocol described in example 1.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone 3 shampoo washes according to the protocol described above. The lower the ΔE value, the more persistent the color with respect to shampoo washing.

The ΔE value is calculated according to the equation described in example 1.

TABLE 12

| | Number of | | | | |
|---|---|---|---|---|---|
| | shampoo | | | | |
| Compositions | washes | L* | a* | b* | ΔE |
| Composition | 0 | 38.0 | 34.9 | 32.5 | — |
| H + I | 3 | 55.0 | 20.6 | 27.0 | 22.9 |
| Composition | 0 | 38.2 | 33.2 | 30.9 | — |
| H + J | 3 | 52.1 | 24.5 | 28.0 | 16.6 |

Thus, the colored coating of the keratin fibers obtained with composition H and composition I according to the invention or with composition H and composition J according to the invention shows good persistence with respect to shampoo washing. Specifically, the locks of hair dyed with composition H and composition I according to the invention or with composition H and composition J according to the invention, and washed with three shampoo washes show good persistence of the color.

1-31. (canceled)

32. A composition for treating keratin fibers, comprising:
a) at least one silicone of formula (I): 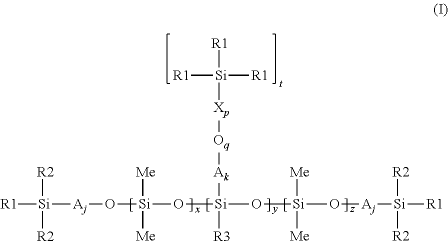
wherein:
R¹ is chosen from a hydroxyl group, an alkoxy group comprising from 1 to 2 carbon atoms, or an alkyl group comprising from 1 to 10 carbon atoms;
R² is chosen from an alkyl group comprising from 1 to 10 carbon atoms, an alkoxy group comprising from 1 to 2 carbon atoms, a hydroxyl group, or a monovalent radical of formula —C_{q}H_{2q}L wherein q is an integer ranging from 2 to 8 and L is an optionally quaternized amino group chosen from the groups:
—N(R″)₂,—N+(R″)₃A⁻,—NR″-Q-N(R″)₂, or —N R″-Q-N+(R″)₃A⁻;
wherein R″ is chosen from a hydrogen atom, a phenyl group, a benzyl group, or a saturated monovalent hydrocarbon-based radical; Q represents a linear or branched group of formula CᵣH₂ᵣ, r is an integer ranging from 2 to 6, and A⁻ represents a cosmetically acceptable anion;
R³ is chosen from a hydroxyl group, an alkyl group comprising from 1 to 10 carbon atoms optionally substituted with at least one group chosen from a hydroxyl group (OH) or a thiol group (SH), a cycloalkyl group comprising from 3 to 20 carbon atoms optionally substituted with at least one group chosen from a hydroxyl group (OH) or a thiol group (SH), an alkoxy group comprising from 1 to 2 carbon atoms optionally substituted with at least one group chosen from a hydroxyl group (OH) or a thiol group (SH), an aryl group comprising from 6 to 12 carbon atoms optionally substituted with at least one group chosen from a hydroxyl group (OH) or a thiol group (SH), a radical —(X)_{p′}—Si(R2)₃, or the monovalent radical of formula —C_{q}H_{2q}L wherein q is an integer ranging from 2 to 8, and L is an optionally quaternized amino group chosen from the groups: —N(R″)₂; —N+(R″)₃ A⁻; —NR″-Q-N(R″)₂, or —NR″-Q-N+(R″)₃ A⁻;
A represents a saturated divalent hydrocarbon-based radical comprising 1 carbon atom;
X represents a hydrogen atom or a saturated, monovalent or divalent hydrocarbon-based radical, comprising 1 carbon atom;
p is an integer ranging from 0 to 6;
p′ is an integer equal to 0, 1, 2, or 3;
k is an integer ranging from 0 to 6;
q is an integer equal to 0 or 1;
j is an integer equal to 0, 1, or 2;
t is an integer equal to 0 or 1;
y is an integer ranging from 0 to 10;
z is an integer ranging from 0 to 500 wherein x+z ranges from 0 to 500 and x+y+z 4;
wherein when X represents a hydrogen atom, then t=0 and p=1;
wherein when p=0, then t=1 and q=1;
wherein at least one of the radicals R¹ or R³ represents a hydroxyl radical, an alkoxy radical comprising from 1 to 2 carbon atoms, or the radical —(X)_{p′}—Si(R2)₃, or the monovalent radical of formula —C_{q}H_{2q}L;
b) at least one silicone of formula (II):
R¹ₐR²_{b}R³_{c}Si O_{(4-a-b-c)/2} (II)
wherein:
R¹ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom or an organic group of formula (A): 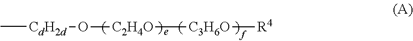
wherein:
R⁴ represents an alkyl group comprising from 1 to 30 carbon atoms or an acyl group comprising from 1 to 30 carbon atoms;
d is an integer ranging from 0 to 15;
e is an integer ranging from 0 to 50; and
f is an integer ranging from 0 to 50;
R² represents a hydroxyl group or an alkoxy group comprising from 1 to 6 carbon atoms, or a group of formula (B): 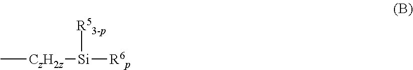
wherein:
R⁵ is chosen from an alkyl group comprising from 1 to 6 carbon atoms, a cycloalkyl group comprising from 5 to 6 carbon atoms, or a phenyl group;
R⁶ represents an alkoxy group comprising from 1 to 6 carbon atoms;
z is an integer ranging from 2 to 8; and
p is an integer ranging from 1 to 3;
R³ represents a silicone compound of formula (C): 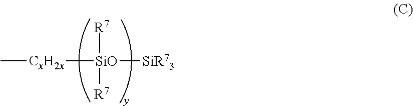
wherein:
R⁷ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom;
x represents an integer ranging from 1 to 5; and
y represents an integer ranging from 0 to 500;
a represents a number ranging from 1.0 to 2.5;
b represents a number ranging from 0.001 to 1.5;
c represents a number ranging from 0 to 1.5; and
wherein when a plurality of R¹ and/or R⁷ radicals are present, the R¹ radicals and R⁷ radicals are independently chosen; and
c) at least one coloring agent chosen from pigments, direct dyes, or mixtures thereof.

33. The composition of claim 32, wherein in the at least one silicone of formula (I):
R¹ is chosen from a hydroxyl group or an alkoxy group comprising from 1 to 2 carbon atoms, or an alkyl group comprising from 1 to 2 carbon atoms;
R² represents an alkyl group comprising from 1 to 4 carbon atoms;
R³ is chosen from a hydroxyl group or an alkyl group comprising from 1 to 10 carbon atoms, an alkoxy group comprising from 1 to 2 carbon atoms or the radical —(X)_{p′}—Si(R²)₃, or a monovalent radical of formula —C_{q}H_{2q}L wherein q is an integer ranging from 2 to 8, and L is an amino group chosen from:
—N(R4)₂; or —N(R4)—CH₂—CH₂—N(R4)₂;
wherein R⁴ represents a hydrogen atom, a phenyl group, a benzyl group, or a saturated monovalent hydrocarbon-based radical;
A represents a saturated divalent hydrocarbon-based radical comprising 1 carbon atom;
X represents a hydrogen atom or a saturated, monovalent or divalent hydrocarbon-based radical comprising 1 carbon atom;
p is an integer ranging from 0 to 6;
p′ is an integer equal to 0, 1, 2, or 3;
j is equal to 0;
k is an integer equal to 0 or 1;
q is an integer equal to 0 or 1;
t is an integer equal to 0 or 1; and
y is an integer ranging from 0 to 10, z is an integer ranging from 0 to 500 wherein x+z ranges from 0 to 500 and x+y+z 4; and
wherein when X represents a hydrogen atom, then t=0 and p=1;
wherein when p=0, then t=1 and q=1; and
wherein at least one of the radicals R¹ or R³ represents a hydroxyl radical or an alkoxy radical comprising from 1 to 2 carbon atoms or the monovalent radical of formula —C_{q}H_{2q}L.

34. The composition of claim 32, wherein in the at least one silicone of formula (I) is chosen from compounds of formula (Ia): 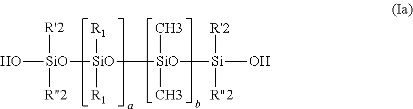
wherein:
R₁ is independently chosen from a hydroxyl group, an alkyl group comprising from 1 to 4 carbon atoms, or an alkoxy group comprising from 1 to 2 carbon atoms;
R′2 and R″2 are independently chosen from an alkyl group comprising from 1 to 4 carbon atoms, or a hydroxyl group; and
a represents an integer ranging from 0 to 10, b represents an integer ranging from 0 to 500 with a+b≥4.

35. The composition of claim 34, wherein the at least one silicone of formula (I) is chosen from compounds of formula (Ia) wherein:
R₁ represents an alkyl group comprising from 1 to 4 carbon atoms;
R′2 and R″2 are independently chosen from alkyl group(s) comprising from 1 to 4 carbon atoms;
a represents an integer ranging from 0 to 10;
b represents an integer ranging from 0 to 500;
wherein a+b≥4.

36. The composition of claim 32, wherein the silicone(s) of formula (I) are present in a total amount ranging from 0.1% to 30% by weight, relative to the total weight of the composition.

37. The composition of claim 32, wherein in the at least one silicone of formula (II):
R¹ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom,
R² is chosen from a hydroxyl group or an alkoxy group comprising from 1 to 6 carbon atoms, or a group of formula (B), wherein R⁶ represents an alkoxy group comprising from 1 to 6 carbon atoms, z is 2, and p is 3;
R³ represents the silicon compound of formula (C), wherein R⁷ is chosen from an alkyl group comprising from 1 to 30 carbon atoms, x is an integer ranging from 1 to 5, and y is an integer ranging from 0 to 500;
a represents a number ranging from 1.0 to 2.5;
b represents a number ranging from 0.001 to 1.5; and
c represents a number ranging from 0 to 1.5.

38. The composition of claim 32, wherein the silicone(s) of formula (II) are present in a total amount ranging from 0.1% to 30% by weight, relative to the total weight of the composition.

39. The composition of claim 32, wherein the composition further comprises at least one alkoxysilane chosen from compounds of formula (III), oligomers thereof, or mixtures thereof:
R¹ₓSi(OR²)₍₄₋ₓ₎ (III)
wherein:
R¹ is chosen from an alkoxy group comprising from 1 to 10 carbon atoms, an amino group NH₂, a linear or branched, saturated or unsaturated, cyclic or acyclic C₁ to C₂₂ hydrocarbon-based radical optionally substituted with at least one group chosen from a hydroxyl group (OH), a thiol group (SH), an alkoxy group comprising from 1 to 10 carbon atoms, an aryl group comprising from 6 to 30 carbon atoms, an amino group NH₂, a (di)alkylamino group NR³R⁴ wherein R³ and R⁴ are independently chosen from a hydrogen atom, a linear or branched alkyl group comprising from 1 to 20 carbon atoms, an aminoalkyl group comprising from 1 to 20 carbon atoms, an aryl group comprising from 6 to 12 carbon atoms or a linear or branched (cyclo)alkyl group comprising from 1 to 20 carbon atoms, wherein R¹ is optionally interrupted with at least one heteroatom chosen from O, S, NH, NR³, a carbonyl group (CO), or combinations thereof;
R² represents a hydrogen atom or an alkyl group comprising from 1 to 20 carbon atoms;
x represents an integer ranging from 1 to 3;
wherein if all the R² radicals represent a hydrogen atom, then R¹ represents an alkoxy group having from 1 to 10 carbon atoms.

40. The composition of claim 39, wherein the at least one alkoxysilane of formula (III), oligomers thereof, or mixtures thereof, is chosen from 3-aminopropyltriethoxysilane (APTES), 3-aminopropylmethyldiethoxysilane (APMDES), N-cyclohexylaminomethyltriethoxysilane, or mixtures thereof.

41. The composition of claim 39, wherein the alkoxysilane(s) of formula (III), oligomers thereof, or mixtures thereof, are present in a total amount ranging from 0.01% to 10% by weight, relative to the total weight of the composition.

42. The composition of claim 39, wherein the composition further comprises at least one metallic compound.

43. The composition of claim 42, wherein the at least one metallic compound comprises at least one metal chosen from titanium, tin, aluminum, zirconium, or mixtures thereof.

44. The composition of claim 42, wherein the at least one organometallic compound is chosen from metal alkoxides of formula (XIXa), wherein:
M represents an atom chosen from titanium, tin, bismuth, aluminum, or zirconium;
n represents the valency of M;
R¹ represents a methyl, ethyl, 2-ethylhexyl, propyl, isopropyl, n-butyl, isobutyl, or t-butyl group.

45. The composition of claim 42, wherein the metallic compound(s) are present in a total amount ranging from 0.01% to 10% by weight, relative to the total weight of the composition.

46. The composition of claim 32, wherein the coloring agent(s) are present in a total amount ranging from 0.001% to 15%, relative to the total weight of the composition.

47. The composition of claim 32, wherein the composition is an anhydrous composition.

48. A method for treating keratin fibers comprising applying to the keratin fibers the composition of claim 32.

49. A method for treating keratin fibers, comprising:
(i) applying to the keratin fibers a composition (A) comprising at least one metallic compound; and
(ii) applying to the keratin fibers a composition (B) comprising:
a) at least one silicone of formula (I): 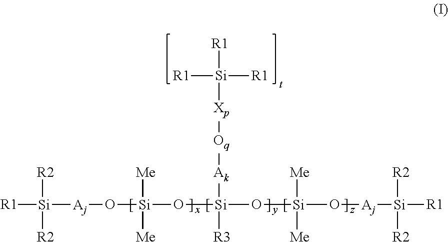
wherein:
R1 is chosen from a hydroxyl group, an alkoxy group comprising from 1 to 2 carbon atoms, or an alkyl group comprising from 1 to 10 carbon atoms;
R2 is chosen from an alkyl group comprising from 1 to 10 carbon atoms, or an alkoxy group comprising from 1 to 2 carbon atoms, a hydroxyl group, or a monovalent radical of formula —C_{q}H_{2q}L wherein q is an integer ranging from 2 to 8 and L is an optionally quaternized amino group chosen from the groups:
—N(R″)₂,—N+(R″)₃A⁻,—NR″-Q-N(R″)₂, or —NR″-Q-N+(R″)₃A⁻;
wherein R″ is chosen from a hydrogen atom, a phenyl group, a benzyl group, or a saturated monovalent hydrocarbon-based radical; Q represents a linear or branched group of formula CᵣH₂ᵣ, r is an integer ranging from 2 to 6, and A⁻ represents a cosmetically acceptable anion;
R3 is chosen from a hydroxyl group, an alkyl group comprising from 1 to 10 carbon atoms optionally substituted with at least one group chosen from a hydroxyl group (OH) or a thiol group (SH), a cycloalkyl group comprising from 3 to 20 carbon atoms optionally substituted with at least one group chosen from a hydroxyl group (OH) or a thiol group (SH), an alkoxy group comprising from 1 to 2 carbon atoms optionally substituted with at least one group chosen from a hydroxyl group (OH) or a thiol group (SH), an aryl group comprising from 6 to 12 carbon atoms optionally substituted with at least one group chosen from a hydroxyl group (OH) or a thiol group (SH), a radical —(X)_{p′}—Si(R²)₃, or the monovalent radical of formula —C_{q}H_{2q}L;
A represents a saturated divalent hydrocarbon-based radical comprising 1 carbon atom;
X represents a hydrogen atom or a saturated, monovalent or divalent hydrocarbon-based radical, comprising 1 carbon atom;
p is an integer ranging from 0 to 6;
p′ is an integer equal to 0, 1, 2, or 3;
k is an integer ranging from 0 to 6;
q is an integer equal to 0 or 1;
j is an integer equal to 0, 1, or 2;
t is an integer equal to 0 or 1;
y is an integer ranging from 0 to 10;
z is an integer ranging from 0 to 500 wherein x+z ranges from 0 to 500 and x+y+z≥4;
wherein when X represents a hydrogen atom, then t=0 and p=1;
wherein when p=0, then t=1 and q=1;
wherein at least one of the radicals R1 or R3 represents a hydroxyl radical, an alkoxy radical comprising from 1 to 2 carbon atoms, or the radical —(X)_{p′}—Si(R2)₃, or the monovalent radical of formula —C_{q}H_{2q}L;
b) at least one silicone of formula (II):
R¹ₐR²_{b}R³_{c}Si O_{(4-a-b-c)/2} (II)
wherein:
R¹ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom or an organic group of formula (A): 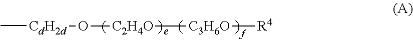
wherein:
R⁴ represents an alkyl group comprising from 1 to 30 carbon atoms or an acyl group comprising from 1 to 30 carbon atoms;
d is an integer ranging from 0 to 15;
e is an integer ranging from 0 to 50; and
f is an integer ranging from 0 to 50;
R² represents a hydroxyl group or an alkoxy group comprising from 1 to 6 carbon atoms, or a group of formula (B): 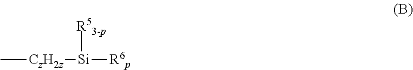
wherein:
R⁵ is chosen from an alkyl group comprising from 1 to 6 carbon atoms, a cycloalkyl group comprising from 5 to 6 carbon atoms, or a phenyl group;
R⁶ represents an alkoxy group comprising from 1 to 6 carbon atoms;
z is an integer ranging from 2 to 8; and
p is an integer ranging from 1 to 3;
R³ represents a silicone compound of formula (C): 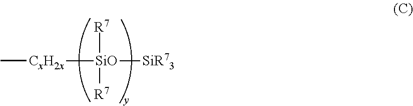
wherein:
R⁷ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom;
x represents an integer ranging from 1 to 5; and
y represents an integer ranging from 0 to 500;
a represents a number ranging from 1.0 to 2.5;
b represents a number ranging from 0.001 to 1.5;
c represents a number ranging from 0 to 1.5; and
wherein when a plurality of R¹ and/or R⁷ radicals are present, the R¹ radicals and R⁷ radicals are independently chosen;
wherein at least one of composition (A) or composition (B) comprises at least one coloring agent chosen from pigments, direct dyes, or mixtures thereof;
wherein steps i) and ii) are performed successively.

50. The method of claim 49, wherein the keratin fibers are washed, rinsed, dried and/or drained after composition (A) is applied to the keratin fibers and before composition (B) is applied to the keratin fibers.

51. A device for treating keratin fibers, comprising at least one compartment comprising:
a first composition comprising:
a) at least one silicone of formula (I): 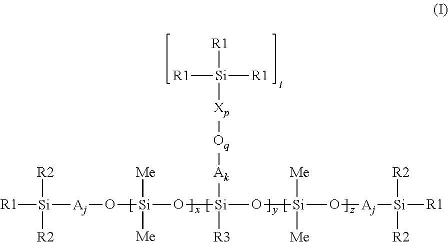
wherein:
R¹ is chosen from a hydroxyl group, an alkoxy group comprising from 1 to 2 carbon atoms, or an alkyl group comprising from 1 to 10 carbon atoms;
R² is chosen from an alkyl group comprising from 1 to 10 carbon atoms, or an alkoxy group comprising from 1 to 2 carbon atoms, a hydroxyl group, or a monovalent radical of formula —C_{q}H_{2q}L wherein q is an integer ranging from 2 to 8 and L is an optionally quaternized amino group chosen from the groups:
—N(R″)₂,—N+(R″)₃ A⁻,—NR″-Q-N(R″)₂, or —NR″-Q-N+(R″)₃A⁻;
wherein R″ is chosen from a hydrogen atom, a phenyl group, a benzyl group, or a saturated monovalent hydrocarbon-based radical; Q represents a linear or branched group of formula CᵣH₂ᵣ, r is an integer ranging from 2 to 6, and A⁻ represents a cosmetically acceptable anion;
R³ is chosen from a hydroxyl group, an alkyl group comprising from 1 to 10 carbon atoms optionally substituted with at least one group chosen from a hydroxyl group (OH) or a thiol group (SH), a cycloalkyl group comprising from 3 to 20 carbon atoms optionally substituted with at least one group chosen from a hydroxyl group (OH) or a thiol group (SH), an alkoxy group comprising from 1 to 2 carbon atoms optionally substituted with at least one group chosen from a hydroxyl group (OH) or a thiol group (SH), an aryl group comprising from 6 to 12 carbon atoms optionally substituted with at least one group chosen from a hydroxyl group (OH) or a thiol group (SH), a radical —(X)_{p′}—Si(R²)₃, or the monovalent radical of formula —C_{q}H_{2q}L: wherein q is an integer ranging from 2 to 8, and L is an optionally quaternized amino group chosen from the groups: —N(R″)₂; —N+(R″)₃ A⁻; —NR″-Q-N(R″)₂, or —NR″-Q-N+(R″)₃ A⁻;
A represents a saturated divalent hydrocarbon-based radical comprising 1 carbon atom;
X represents a hydrogen atom or a saturated, monovalent or divalent hydrocarbon-based radical, comprising 1 carbon atom;
p is an integer ranging from 0 to 6;
p′ is an integer equal to 0, 1, 2, or 3;
k is an integer ranging from 0 to 6;
q is an integer equal to 0 or 1;
j is an integer equal to 0, 1, or 2;
t is an integer equal to 0 or 1;
y is an integer ranging from 0 to 10;
z is an integer ranging from 0 to 500 wherein x+z ranges from 0 to 500 and x+y+z≥4;
wherein when X represents a hydrogen atom, then t=0 and p=1;
wherein when p=0, then t=1 and q=1;
wherein at least one of the radicals R1 or R3 represents a hydroxyl radical, an alkoxy radical comprising from 1 to 2 carbon atoms, or the radical —(X)_{p′}—Si(R2)₃, or the monovalent radical of formula —C_{q}H_{2q}L;
b) at least one silicone of formula (II):
R¹ₐR²_{b}R³_{c}Si O_{(4-a-b-c)/2} (II)
wherein:
R¹ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom or an organic group of formula (A): 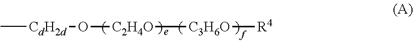
wherein:
R⁴ represents an alkyl group comprising from 1 to 30 carbon atoms or an acyl group comprising from 1 to 30 carbon atoms;
d is an integer ranging from 0 to 15;
e is an integer ranging from 0 to 50; and
f is an integer ranging from 0 to 50;
R² represents a hydroxyl group or an alkoxy group comprising from 1 to 6 carbon atoms, or a group of formula (B): 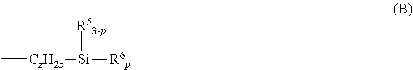
wherein:
R⁵ is chosen from an alkyl group comprising from 1 to 6 carbon atoms, a cycloalkyl group comprising from 5 to 6 carbon atoms, or a phenyl group;
R⁶ represents an alkoxy group comprising from 1 to 6 carbon atoms;
z is an integer ranging from 2 to 8; and
p is an integer ranging from 1 to 3;
R³ represents a silicone compound of formula (C): 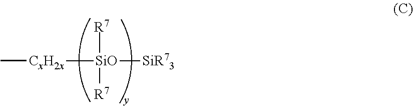
wherein:
R⁷ is chosen from an alkyl group comprising from 1 to 30 carbon atoms, a cycloalkyl group comprising from 5 to 10 carbon atoms, an aryl group comprising from 6 to 10 carbon atoms, an aralkyl group comprising from 7 to 14 carbon atoms, or an alkyl group comprising from 3 to 10 carbon atoms substituted with at least one fluorine atom;
x represents an integer ranging from 1 to 5; and
y represents an integer ranging from 0 to 500;
a represents a number ranging from 1.0 to 2.5;
b represents a number ranging from 0.001 to 1.5;
c represents a number ranging from 0 to 1.5; and
wherein when a plurality of R¹ and/or R⁷ radicals are present, the R¹ radicals and R⁷ radicals are independently chosen; and
c) at least one coloring agent chosen from pigments, direct dyes, or mixtures thereof;
and
optionally, a second compartment comprising a composition (G) comprising at least one alkoxysilane chosen from the compounds of formula (III), oligomers thereof, or mixtures thereof:
R¹ₓSi(OR²)₍₄₋ₓ₎ (III)
wherein:
R¹ is chosen from an alkoxy group comprising from 1 to 10 carbon atoms, an amino group NH₂, a linear or branched, saturated or unsaturated, cyclic or acyclic C₁ to C₂₂ hydrocarbon-based radical optionally substituted with at least one group chosen from a hydroxyl group (OH), a thiol group (SH), an alkoxy group comprising from 1 to 10 carbon atoms, an aryl group comprising from 6 to 30 carbon atoms, an amino group NH₂, a (di)alkylamino group NR³R⁴ wherein R³ and R⁴ are independently chosen from a hydrogen atom, a linear or branched alkyl group comprising from 1 to 20 carbon atoms, an aminoalkyl group comprising from 1 to 20 carbon atoms, an aryl group comprising from 6 to 12 carbon atoms or a linear or branched (cyclo)alkyl group comprising from 1 to 20 carbon atoms, wherein R¹ is optionally interrupted with at least one heteroatom chosen from O, S, NH, NR³, a carbonyl group (CO), or combinations thereof;
R² represents a hydrogen atom or an alkyl group comprising from 1 to 20 carbon atoms;
x represents an integer ranging from 1 to 3;
wherein if all the R² radicals represent a hydrogen atom, then R¹ represents an alkoxy group having from 1 to 10 carbon atoms.
